(19) United States
(12) Patent Application Publication
(10) Pub. No.: US 2011/0213288 A1
(43) Pub. Date: Sep. 1, 2011

(54) Device And Method For Transfecting Cells For Therapeutic Uses

(75) Inventors: Yoonsu Choi, Houston, TX (US); Lawrence J.N. Cooper, Houston, TX (US); Dean A. Lee, Pearland, TX (US); Sibani Lisa Biswal, Houston, TX (US); Robert Raphael, Houston, TX (US); Thomas C. Killian, Houston, TX (US)

(73) Assignees: The Board of Regents, The University of Texas System, Austin, TX (US); William Marsh Rice University, Houston, TX (US)

(21) Appl. No.: 12/884,084

(22) Filed: Sep. 16, 2010

Related U.S. Application Data
(63) Continuation-in-part of application No. 12/674,151, filed on Jun. 24, 2010.
(60) Provisional application No. 60/925,830, filed on Apr. 23, 2007.

(51) Int. Cl. A61M 1/36; C12M 1/42; C12N 13/00

(52) U.S. Cl. 604/6.08; 435/283.1; 435/173.6

(57) ABSTRACT

This invention generally relates to devices and methods for ex vivo or in vivo transfection of living cells using electroporation, in particular high throughput microfluidic electroporation, and to therapeutic uses of the transfected cells.

### RELATED APPLICATIONS

This application is a continuation-in-part of U.S. patent application Ser. No. 12/674,151, filed 24 Jun. 2010, which is incorporated by reference, including any figures, as if fully set forth herein, and which claims the benefit of PCT Patent Application No. US2008/061342, filed 23 Apr. 2008, and which claims the benefit of U.S. Provisional Patent Application No. 60/925,830, filed on 23 Apr. 2007.

### STATEMENT AS TO FEDERALLY SPONSORED RESEARCH

This invention was made in part with Government funding and the Government therefore has certain rights in the invention.

### FIELD

This invention relates to molecular biology, physics, microfabrication, microfluidics, genetic material therapy and medicine. In particular, it relates to devices and methods for stable and transient insertion of therapeutic nucleic acids into mammalian cells by electroporation and use of the transfected cells in the treatment diseases.

### BACKGROUND

There is a current trend to produce micro- and nano-scale devices that can perform physical, chemical, and biological processes on a small scale with the same efficiency as conventional macroscopic systems. These micro total analytical systems (μTAS) provide sample handling, separation, and detection on a single device using miniscule sample and reagent volumes. In fact, a variety of micro components such as pumps, valves, mixers, filters, heat exchangers, and sensors have been developed and used to create “lab-on-a-chip” devices.

Another current trend in the medical field has been development of cell-based therapy for the treatment of diseases. In its most basic manifestation, cell-based therapy involves the alteration of the genome of living cells whereby faulty genes that either do not express an essential protein or express a mutant protein, which may be non-functional or may function abnormally to produce a particular disease, are “repaired.” Since the genome itself is affected, the repaired gene will be passed on to daughter cells. The as of yet unfulfilled goal of gene therapy is the treatment of genetic diseases such as cystic fibrosis, Down syndrome, Huntington's disease, dwarfism, sickle cell anemia, Tay-Sachs disease, phenylketonuria, amyotrophic lateral sclerosis (ALS, Lou Gehrig's disease), Parkinson's disease and many others. This type of cell-based therapy is formally termed “gene therapy,” because it is so defined by the FDA: “ . . . a medical intervention based on modification of genetic material of living cells. Cells may be modified ex vivo for subsequent administration or may be altered in vivo by gene therapy products given directly to the subject.”

An alternative to gene therapy is transient transfection of nucleic acids coding for desired proteins into cells where the proteins are either expressed on the cells' surface to direct or redirect the cells responsiveness to outside influences or are secreted by the cells to provide therapeutic biologics.

While there is considerable cross-over among the techniques for effecting gene therapy and transient transfection, the most prevalent procedure for the former is by means of vectors such as viruses, retroviruses, adenoviruses, adeno-associated viruses and the like. While viral gene transfection is extremely efficient, it is not without significant problems such as toxicity and other undesired side effects, difficulty in assuring the virus infects the correct target cell, ensuring that the inserted gene does not disrupt any other genes, etc. Transient transfection, since it does not involve interaction with the genome, circumvents many of the problems.

Transient transfection may be accomplished by a variety of mechanical, chemical and electrical means. Mechanical means of transfection include direct microinjection, particle bombardment with DNA-gold microarticles and pressured infusion. Chemical transfection involves the use of agents capable of disrupting the plasma membrane sufficiently to permit exogenous materials such as therapeutic agents to cross.

Chemical transfection agents include DEAE dextran, calcium phosphate, polyethylenimine and lipids. A fundamental problem with chemical transfection is toxicity; it has been posited that there is no chemical agent that doesn't have some toxic effect on cells.

Electrical techniques for transfection are dominated by electroporation, which involves application of a high electric field to the cells, which causes disruption of the phospholipid bilayer of the plasma membrane resulting in the formation of pores in the membrane through which extracellular materials can pass. Since the electric potential across cell membrane rises about 0.5 to 1.0 volt concurrently with the formation of pores, charged molecules such as DNA are driven through the pores in a manner similar to electrophoresis. On removal of the electric field the membrane quickly reseals leaving the cells intact. Electroporation can be accomplished by batch-processing cells in cuvettes or on multiwell plates and, more recently, using microfluidics. None of these methods as currently practiced is particularly amenable to mass production of transfected cells in clinically useful quantities except through propagation of the transfected cells to prepare the required number of cells.

Thus, as currently practiced, all cell transfection techniques, those mentioned above as well as the many others known to those skilled in the art are extremely labor intensive, inefficient, and typically rely on access to full-scale good manufacturing practice (GMP) facilities, biological safety level 2 (BSL2) at least, which renders them prohibitively expensive.

What is needed is an efficient, economic means of transfecting cells in therapeutically useful quantities and subsequently administering those cells to patients in need thereof, all in a clinical setting.

### SUMMARY

Thus, an aspect of this invention is directed to a device, comprising: a base unit having a top surface and a bottom surface essentially parallel to and opposite the top surface;
a first reaction tier comprising a plurality of microfluidic chambers impressed into the base unit, each chamber being defined by one or more side walls and a floor and having dimensions that permit the chamber to hold one intact eukaryotic cell; wherein:
each chamber has a port extending from approximately the center of the floor of the chamber to the bottom surface of the base unit, where the port is capable of fluidic connection with an external source;
each chamber has one or more additional ports extending from the floor of the chamber to the bottom surface of the base unit, where each additional port is individually capable of fluidic connection with an external source;
each chamber has a positive electrode and negative electrode operatively coupled to its wall(s) wherein the electrodes are disposed substantially opposite one another.

In an aspect of this device, the plurality of microfluidic chambers is divided into arrays of two or more chambers each.

In an aspect of this device, the center port is operatively coupled to a negative pressure device.

In an aspect of this device, each port is separated from the chamber by a diffusion barrier.

In an aspect of this device, the diffusion barrier comprises a mesh having pores about 1 μm in diameter.

In an aspect of this device, the eukaryotic cell is a primary human T cell.

In an aspect of this device, each chamber has a volume of about 8000 μm³.

In an aspect of this device, the arrays of microfluidic chambers are subdivided into two or more subarrays by a wall that surrounds and fluidically separate each subarray from each other subarray thereby forming a second reaction tier.

In an aspect of this device, the height of the raised wall separating the subarrays is about ten times the height of a chamber wall.

An aspect of this device comprises a second raised wall enclosing all of the subarrays thereby forming a third reaction tier.

In an aspect of this device, the second raised wall has a wall height of about 2 mm to about 5 mm.

In an aspect of this device, each array comprises 9 chambers.

In an aspect of this device, each subarray comprises 9 arrays.

In an aspect of this device, the total number of chambers is 324.

An aspect of this device is a method of transfecting eukaryotic cells with non-integrating mRNA, comprising:

introducing a plurality of eukaryotic cells into a device hereof;
applying a negative pressure through the center port in each chamber;
manipulating the device and cells until one cell enters each chamber and is held there by the applied negative pressure;
removing excess cells;
introducing an electroporation buffer into each chamber;
applying a voltage across the electrodes in each chamber;
introducing an mRNA reagent into each chamber through one of the additional ports in each chamber wherein the mRNA being introduced into each chamber may be the same as or different from the mRNA being introduced into each other chamber;
turning off the voltage across each chamber after a predetermined time;
removing the mRNA reagent from each chamber;
washing the cell in each chamber;
introducing one or more second reagent(s) into each chamber through one or more of the additional ports in each chamber wherein the second reagent(s) being introduced into each chamber may be the same as or different than the second reagent being introduced into each other chamber;
removing the second reagent(s) from each chamber after a second predetermined time;
washing the cells in each chamber;
releasing the negative pressure in those chambers containing similarly treated cells;
optionally applying a positive pressure into each chamber in which the negative
pressure has been released through the center port of each chamber;
collecting the released cells; and,
repeating the release of negative pressure and optional application of positive pressure sequentially in chambers holding additional groups of similarly treated cells and collecting the groups of similarly treated cells until all the cells have been collected.

In an aspect of this device, the above method further comprises: introducing one or more third reagent(s) into the second reaction tier sub-arrays after removing the second reagent(s) and washing the cells wherein the third reagent(s) introduced into each sub-array may be the same as or different from the third reagent introduced into each other sub-array;

removing the third reagent(s) from the sub-arrays after a third predetermined time;
washing the cells in each chamber;
releasing the negative pressure in those chambers containing cells similarly treated in both the first and second reaction tiers;
optionally applying a positive pressure into each chamber in which the negative pressure has been released through the center port of each chamber;
collecting similarly treated cells; and
repeating the release of negative pressure and optional application of positive pressure sequentially in chambers holding additional groups of similarly treated cells and collecting the groups of similarly treated cells until all the cells have been collected.

In an aspect of this invention, the above method further comprises:

Introducing one or more fourth reagent(s) into the third reaction tier after washing the cells;
removing the fourth reagent(s) from the third reaction tier after a fourth predetermined time;
washing the cells in each chamber;
releasing the negative pressure in those chambers containing cells similarly treated in the first, second and third reaction tiers;
optionally applying a positive pressure into each chamber in which the negative pressure has been released through the center port of each chamber;
collecting similarly treated cells; and
repeating the release of negative pressure and optional application of positive pressure sequentially in chambers holding additional groups of similarly treated cells and collecting the groups of similarly treated cells until all the cells have been collected.

An aspect of this invention relates to a device comprising:

an orifice plate having an inlet surface, an outlet surface and an outer edge having a thickness;
one or more through-holes extending through the orifice plate from the inlet surface to the outlet surface, the surface between the inlet and outlet surfaces comprising a wall surface; wherein
each through-hole is sized to permit a single eukaryotic cell at a time to pass through;
each through-hole has a positive electrode operatively coupled to its wall surface substantially opposite a negative electrode likewise operative coupled to its wall surface;
   a positive electrode connection and an negative electrode connection operatively coupled to the outer edge of the orifice plate, the positive electrode connection being operatively coupled to each positive electrode in each through-hole and the negative electrode connection being operatively coupled to each negative electrode in each through-hole;
   an inlet exterior source connector operatively coupled to the inlet surface of the orifice plate; and
   an outlet connector operatively coupled to the outlet surface of the orifice plate.

In an aspect of this invention, the above device further comprises two or more external sources operatively coupled to the inlet exterior course connector.

In an aspect of this invention, with regard to the above device, one external source is a source of eukaryotic cells and another external source is a source of a non-integrating nucleic acid.

In an aspect of this invention, with regard to the above device the eukaryotic cells are primary human T-cells.

In an aspect of this invention the non-integrating nucleic acid is non-integrating mRNA.

In an aspect of this invention, the outlet connector is operatively coupled to a collection device.

In an aspect of t his invention, the above device further comprises a u-shaped electrical connection device comprising a base and two side parallel side walls, one side wall having a positive pole electrical contact operatively coupled to a positive pole of an external voltage source and the other side wall having a negative pole electrical contact operatively coupled to a negative pole of the external voltage source, wherein
the side walls are spaced apart such that when the orifice plate is placed between them the positive electrode connection makes electrical contact with the positive pole electrical contact on one wall of the U-shaped device and the negative electrode connection makes electrical contact with the negative pole electrical contact on the opposite wall of the U-shaped device.

An aspect of this invention relates to a method of treating a disease, comprising:

identifying a subject afflicted with a disease that is known to be, becomes known to be or is suspected of being responsive to treatment using transfected cells;
inserting a sterile needle that is operatively coupled to a cell separator that in turn is operatively coupled to the inlet exterior source connector or the device of claim 17 into a blood vessel of a subject;
withdrawing blood from the subject and transporting it through sterile tubing to the cell separator wherein cells of a type that is to be electro-transfected are selected and separated from other cell types in the blood;
introducing the selected cells along with a non-integrating nucleic acid to the input surface side of the orifice plate and then passing the mixture through the through-holes in the orifice plate in which through-holes a voltage has been created using the external voltage source such that the cells are electroporated and transfected as they pass through;
transporting the transfected cells through the outlet connector, which has been operatively connected to a sterile syringe needle that has been inserted into a blood vessel of the subject, back into the subject.

In an aspect of this invention, with regard to the above method the subject is a mammal.

In an aspect of this invention, the mammal is a human being.

In an aspect of this invention, the human being is a pediatric patient.

In an aspect of this invention, the selected cell type is selected from the group consisting of T cells, NK cells, B cells, dendritic (antigen presenting) cells, monocytes, reticulocytes, stem cells, tumor cells, umbilical cord blood-derived cells, peripheral-blood derived cells and combinations thereof.

In an aspect of this invention, the stems cells are selected from the group consisting of hematopoietic stem cells and mesenchymal stem cells.

In an aspect of this invention, the selected cell type is selected from the group consisting of T cells, NK cells or a combination thereof.

In an aspect of this invention, the selected cell type is primary human t-cells.

In an aspect of this invention, the non-integrating nucleic acid is a non-integrating RNA.

In an aspect of this invention, the non-integrating RNA is selected from the group consisting of mRNA, microRNA and siRNA.

In an aspect of this invention, the non-integrating RNA codes for a biotherapeutic agent.

In an aspect of this invention, the biotherapeutic agent is selected from the group consisting of a chimeric antigen receptor, an enzyme, a hormone, an antibody, a clotting factor, a Notch ligand, a recombinant antigen for vaccine, a cytokine, a cytokine receptor, a chemokine, a chemokine receptor, an imaging transgene, a co-stimulatory molecule, a T-cell receptor, FoxP3, a luminescent probe, a fluorescent probe, a reporter probe for positron emission tomography, a sodium iodine symporter, a KIR deactivator, hemoglobin, an Fc receptor, CD24, BTLA, a transposase, a transposon, a transposon from Sleeping Beauty or piggyback and combinations thereof.

In an aspect of this invention, the disease is selected from the group consisting of a pathogenic disorder, cancer, enzyme deficiency, in-born error of metabolism, infection, auto-immune disease, obesity, cardiovascular disease, neurological disease, neuromuscular disease, blood disorder, clotting disorder and a cosmetic defect.

### DETAILED DESCRIPTION

### Brief Description of the Drawings

The drawings herein are provided for the sole purpose of aiding in the understanding of this invention; they are in no manner intended nor should they be construed as limiting the scope of this invention in any manner whatsoever.

FIG. 1 shows a DNA plasmid vector which serves as the in vitro template for translation.

FIG. 2 shows formaldehyde-agarose gel electroporation of in vitro transcribed CD19R and CD19RCD28 mRNAs. These mRNAs code for a chimeric antigen receptor with specificity for CD19.

FIG. 3A shows a FACS analysis of Jurkat cells (T cell), NK92 cells (NK cells) electroporated with CD19R and CD19RCD28 mRNAs synthesized from the vectors. Cells were analyzed with 2D3 Alexa-labeled CD19R-specific mAb (made at MDACC, Houston Tex.) and NK-cell marker CD56. Propidium iodide (PI) staining was used to determine the viability of the cells after electroporation.

FIG. 3B shows the determination of the fate of mRNA in cells after electroporation as determined by Cy5-labeled CD19R mRNA as well as 2D3 Alexa-labeled CD19R-specific antibody.

FIG. 4 is a FACS analysis of OKT-3/IL-2 activated T-cells and Jurkat cells electroporated with CD19RCD28 mRNAs synthesized from the T7 based CD19RCD28 plasmid vectors.

FIG. 5 is a schematic illustration of an embodiment of the present invention for creating a focused stream of single cells using microfluidics.

FIG. 6 shows a side view of a cell traveling through multiple electric fields to improve transfection efficiency.

FIG. 7 shows detection of cell transfection by an embodiment of the present invention using fluorescence.

FIG. 8 shows a summary of a clinical trial design for an embodiment of the non-integrating method described herein.

FIG. 9 shows a schematic representation of biodistribution of infused therapeutic agents as derived by NIP technology.

FIG. 10 shows phenotype and function of genetically modified T cells.

FIG. 11 shows binding of anti-CD20-IL-2 ICK to B cells and T cells.

FIG. 12 shows effect of ICK on persistence of adoptively transferred T cells.

FIG. 13 shows combined anti-tumor efficacy of ICK and CD19-specific T cells.

FIG. 14 shows measurement of both T-cell persistence and anti-tumor effect of immunotherapies in individual mice.

FIG. 15 shows a microfluidic electroporation unit of this invention.

FIG. 16 shows one embodiment of a “GMP-in-a-box” of this invention wherein the microfluidic electroporation unit of FIG. 15 is encased in a housing that can comprise a disposable cartridge.

FIG. 17 shows a number of the microfluidic electroporation units of FIG. 15 arrayed in housing such that the device and method thereof is capable of high throughput operation.

FIG. 18 shows a microfluidic electroporation unit of this invention sized down to be implantable in the body of a patient.

FIG. 19A shows a single microfluidic chamber embodiment of this invention.

FIG. 19B shows a side view of the microfluidic chamber of FIG. 19A in which a center port and two side ports are shown. The end of each port at the bottom of the base is adapted to be the female portion of a fluidic coupling with an external source.

FIG. 19C shows a side view of the microfluidic chamber of FIG. 19A in which a center port and two side ports are shown. The end of each port at the bottom is extended to from the male portion of a fluidic coupling with an external source.

FIG. 20A shows a second single microfluidic chamber embodiment of this invention.

FIG. 20B shows a side view of the microfluidic chamber of FIG. 20A in which a center port and two side ports are shown. The end of each port at the bottom of the base is adapted to be the female portion of a fluidic coupling with an external source.

FIG. 20C shows a side view of the microfluidic chamber of FIG. 20A in which a center port and two side ports are shown. The end of each port at the bottom is extended to form the male portion of a fluidic coupling with an external source.

FIG. 21 shows a top and side view of an array of microfluidic chambers of FIG. 19 or 20 in a base unit, top and side view.

FIG. 22 shows a top and side view of the array of microfluidic chambers of FIG. 21, wherein the array has been divided into a plurality of subarrays, each subarray being fluidically separated from each other subarray.

FIG. 23 shows a top and side view of the subarrays of FIG. 22 surrounded completely by a wall that permits fluidic contact of all chambers.

FIG. 24 shows two views of a microfluidic electroporation unit as described herein, having a size that can accommodate enough electroporation activity to provide sufficient numbers of cells for clinical applications. The device comprises an orifice plate combined with electrodes, and two connecting adapter tubes.

FIG. 25 shows the device of FIG. 24 being installed into a holder in the system. As can be seen in the semi-transparent image, an orifice plate is aligned with the positive and negative electrodes of the holder.

### DETAILED DESCRIPTION

It is understood that with regard to this description and the appended claims, any reference to any aspect of this invention made in the singular includes the plural and vice versa unless it is expressly stated or unambiguously clear from the context that such is not intended.

As used herein, any term of approximation such as, without limitation, near, about, approximately, substantially, essentially and the like mean that the word or phrase modified by the term of approximation need not be exactly that which is written but may vary from that written description to some extent. The extent to which the description may vary will depend on how great a change can be instituted and have one of ordinary skill in the art recognize the modified version as still having the properties, characteristics and capabilities of the modified word or phrase. In general, but with the preceding discussion in mind, a numerical value herein that is modified by a word of approximation may vary from the stated value by ±15%, unless expressly stated otherwise.

As used herein, “optional” means that the element modified by the term may or may not be present.

As used herein, the terms “preferred,” “preferably,” and the like refer to the situation as it existed at the time of filing this patent application.

As used herein, “high throughput” refers to the production of a sufficient number of transfected cells to be therapeutically effective in a clinically relevant time-frame. To be therapeutically effective the transfected cells must produce a selected biotherapeutic agent in sufficient quantity to have a beneficial effect on the health and well-being of a patient being treated. A beneficial effect on the health and well-being of a patient includes, but is not limited to: (1) curing the disease; (2) slowing the progress of the disease; (3) causing the disease to retrogress; or, (4) alleviating one or more symptoms of the disease. As used herein, a biotherapeutic agent also includes any substance that when administered to a patient, known or suspected of being particularly susceptible to a disease, in a prophylactically effective amount, has a prophylactic beneficial effect on the health and well-being of the patient. A prophylactic beneficial effect on the health and well-being of a patient includes, but is not limited to: (1) preventing or delaying on-set of the disease in the first place; (2) maintaining a disease at a retrogressed level once such level has been achieved by a therapeutically effective amount of a substance, which may be the same as or different from the substance used in a prophylactically effective amount; or, (3) preventing or delaying recurrence of the disease after a course of treatment with a therapeutically effective amount of a substance, which may be the same as or different from the substance used in a prophylactically effective amount, has concluded.

As used herein, A “fluidic connection” simply refers to a connection between two elements of the device herein where, if the elements are said to be in “fluidic connection”, this means that a fluid, which may be a gas or a liquid which may contain substances dissolved or suspended in them, will easily flow from one such element to all others with which it is in fluidic connection. To the contrary, if elements of this invention as said to not be in fluidic connection, fluids together with whatever may be dissolved or suspended in them cannot flow from one element to another.

As used herein, an “external source” refers to a reservoir of a fluid that is separate from the device of this invention but is capable of forming a fluidic connection with an element of the invention. In particular, the ports of the invention are designed and constructed so as to be connected to an external source so that fluids contained in the external source reservoir can be supplied to various elements, e.g., chambers, arrays of chamber, etc. of the device.

As used herein, “microfluidic” retains the meaning that would be understood by those skilled in the art; that is, in general it refers to a device that has one or more channels with at least one dimension less than 1 mm. The devices of the current invention have a dimension, the distance between two substantially parallel conductive surfaces that is no more than about 100 μm, preferably no more than about 50 μm and thus qualifies as microfluidic. With regard to “microfluidic chambers” of this invention, such refers to chambers, wells, depressions, etc. impressed into the top surface of a base unit wherein the chambers have length, width and height dimensions that are all less than 1 mm. In a presently preferred embodiment of this invention, the dimensions of a microfluidic chamber of this invention has dimensions of about 20 μm×20 μm×20 μm or 8,000 μm³, which clearly also qualifies as “microfluidic.” It is understood that the phrase “impressed into the surface” when referred to the microfluidic chambers of this invention is not intended and is not to be construed as in any manner limiting the technique used to make the chambers. They can indeed be impressed into the surface by application of pressure to a suitably deformable base unit material or they can be, without limitation, drilled or laser cut into the surface. Any means of creating such chambers is within the scope of this invention.

As used herein, “electroporation,” “electroporating” and other versions of the word likewise have the meaning generally ascribed to them by those skilled in the art and therefore will not be described at length or in depth here. Those skilled in the art understand the technology and procedures extremely well and those techniques and procedures are applicable to the invention herein. In any event, in brief, electroporation refers to the process of subjecting a living cell to an electric field such that, when the voltage across the plasma membrane of the cell exceeds its dielectric strength, the membrane is disrupted and pores form in it through which substances, in particular polar substances that normally are unable to traverse the membrane, can pass and enter the cytoplasm of the cell. If the strength of the electric field coupled with the time of exposure is properly selected, the pores reseal after the cell is removed from the electric field.

Electroporation buffers are a well-known aspect of the art of electroporation and likewise need no extensive description as they are very well known in the art as are procedures for determining which buffer is optimal for use with a particular cell type and particular substance, such as herein, mRNA, that is to be electro-transferred into the cells. Any electroporation buffer presently known in the art, such as, without limitation, commercial buffers offered by Amaxa Biosystems as well as any electroporation buffers that may become known in the future may be used with the device of this invention; such use is within the scope of this invention.

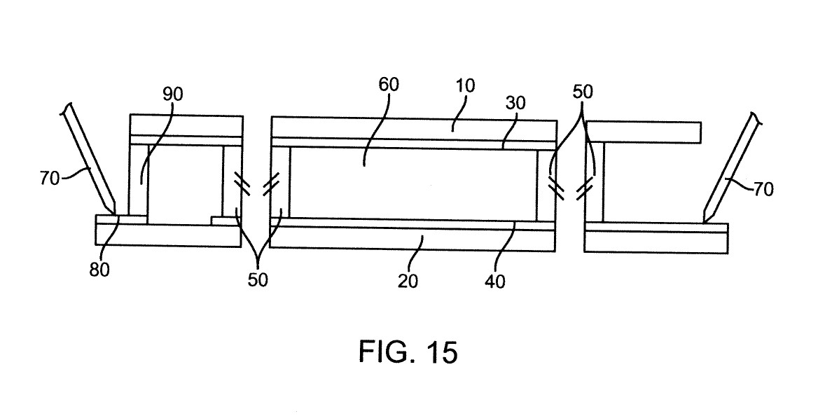

As used herein, an “electroporation unit” refers to all of the elements of a device necessary to cause the high throughput electroporation of living cells. A diagram of an exemplary but non-limiting electroporation unit of the current invention is shown in FIG. 15. In FIG. 15, the view is looking down a channel of the device from a proximal end of the device to a distal end of the device. Only a single channel is shown whereas the device may comprise a large number of parallel channels. In FIG. 15, non-conductive support elements 10 and 20 are made of any type of material having sufficient mechanical strength to maintain the mechanical integrity of the unit. They may be made of such material as a glass including without limitation Pyrex®, a ceramic, a non-conductive polymer, a mineral such as sapphire. It is presently preferred that the support elements be made of a biocompatible substance, that is a substance that will not have a deleterious effect on cells and other biological substances that might come in contact with the element. Support elements 10 and 20 are coated with conductive layers 30 and 40. Conductive layers 30 and 40 can be made of any conductive biocompatible material such as, without limitation, a biocompatible conductive metal such as, without limitation, gold, or a biocompatible conductive polymer. They may be applied to the surfaces of the support elements by any means known or as becomes known in the art for accomplishing such including, without limitation, microlithography, vapor deposition, plasma deposition, and the like. If the conductive layer material does not adhere to the surface of the support elements, a primer layer to which the conductive material will adhere may be first applied to the support surfaces. The distance between the conductive surfaces is maintained by a plurality of non-conductive spacers 50 that extend essentially the full length of the conductive layers and are contiguous with the layers so as to form a number of discrete channels 60 in the unit. The non-conductive spacers, like the non-conductive support elements, can be made of any non-conductive material capable of maintaining the mechanical integrity of the structure such as, without limitation, a non-conductive polymer. The distance between the conductive surfaces as established by the spacers is not greater than about 100 μm, preferably at present not more than about 50 μm. The distance between spacers can be any that is desired. Finally, the electroporation unit comprises a pulse generator that is in electrical contact with the conductive surfaces, one lead of the generator being in contact with each of the conductive surfaces. As depicted in FIG. 15., electrical contact is made using Pogo® pins 70, which are well known by those skilled in the microelectronics art. The right hand pin is in contact with conductive layer 40 while the left hand pin is in contact with conductive material 80, which may be the same as or different than conductive layers 30 and 40 and conductive material 80 is in electrical contact with conductive vertical element 90 that, in turn, is in electrical contact with the conductive layer 30. The ends of the pins that are not shown in contact with the device are of, course, connected to the pulse generator.

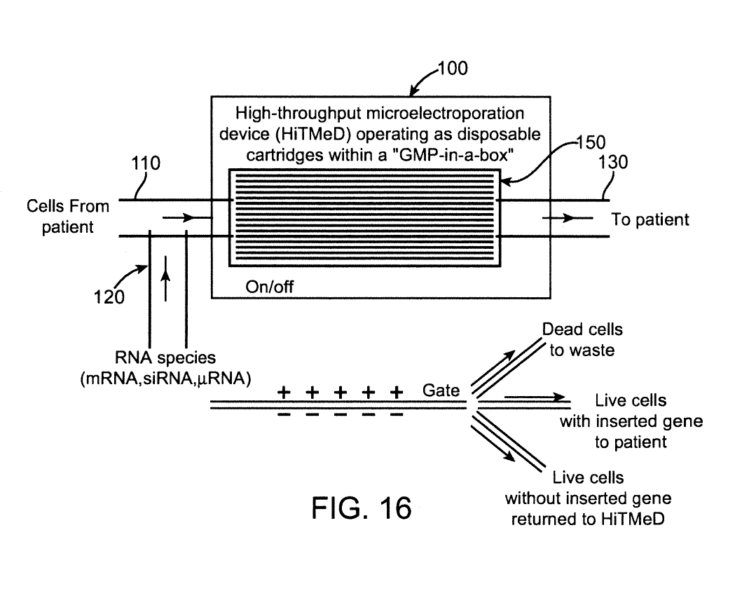

In some embodiments, the microfluidic electroporation units (MEU) described herein may be used individually as illustrated in FIG. 16. In FIG. 16 MEU 105 is contained in a sealable sterile housing 100, which may be reusable, or a disposable cartridge. The patient is the source of cells to be transformed as is shown in FIG. 16, the inlet 110 labeled “cells from patient.” The cells are collected from the patient by tapping a selected source of bodily fluid such as, without limitation, venipuncture of a vein from which blood is drawn. Other sources include an indwelling catheter or a central intravenous catheter. Being mixed with the cells from the patient prior to their entry into the MEU is a stream of an RNA species from inlet 120 with which the cells will be transformed. Inlet 120 is shown in FIG. 16 as being outside the housing or cartridge; however, it may be connected to the housing itself such that the cells and the RNA mix inside the housing just prior to electroporation. Once the cells have been electroporated and the RNA has entered the cells, the transformed cells exit the MEU and the housing through outlet 130 and are returned to the patient through the same or a different route, i.e., the same venipuncture that was used to collect the cells in the first place or they may be returned by means of a separate venipuncture. If desired, transformed cells can be separated from living-but-not-transformed and from dead cells as shown in the second diagram of FIG. 16. The cell separation component may be external to housing 100 or it may be internal so as to render the entire apparatus as self-contained as possible.

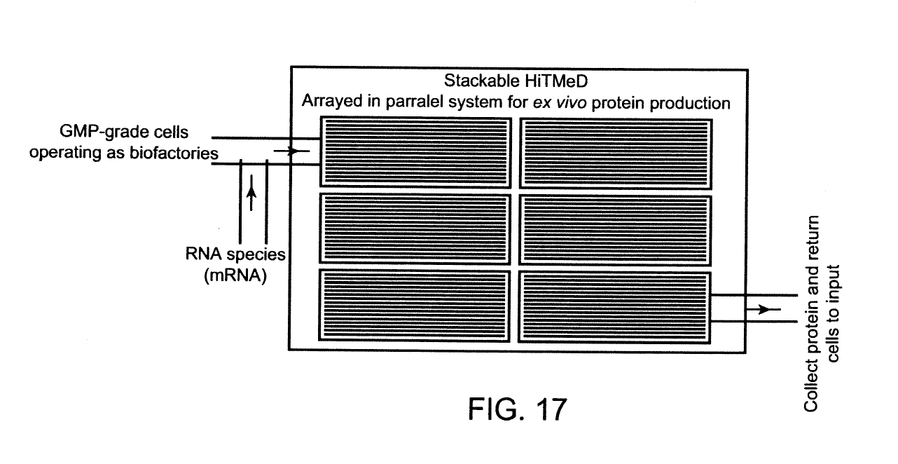

While MEUs may be used individually as shown in FIG. 16, preferably at present they may be used in arrays of multiple MEUs to facilitate high throughput transfection of cells and enhance the therapeutic utility of the devices and methods of this invention. A non-limiting schematic of stacked MEU units is shown in FIG. 17.

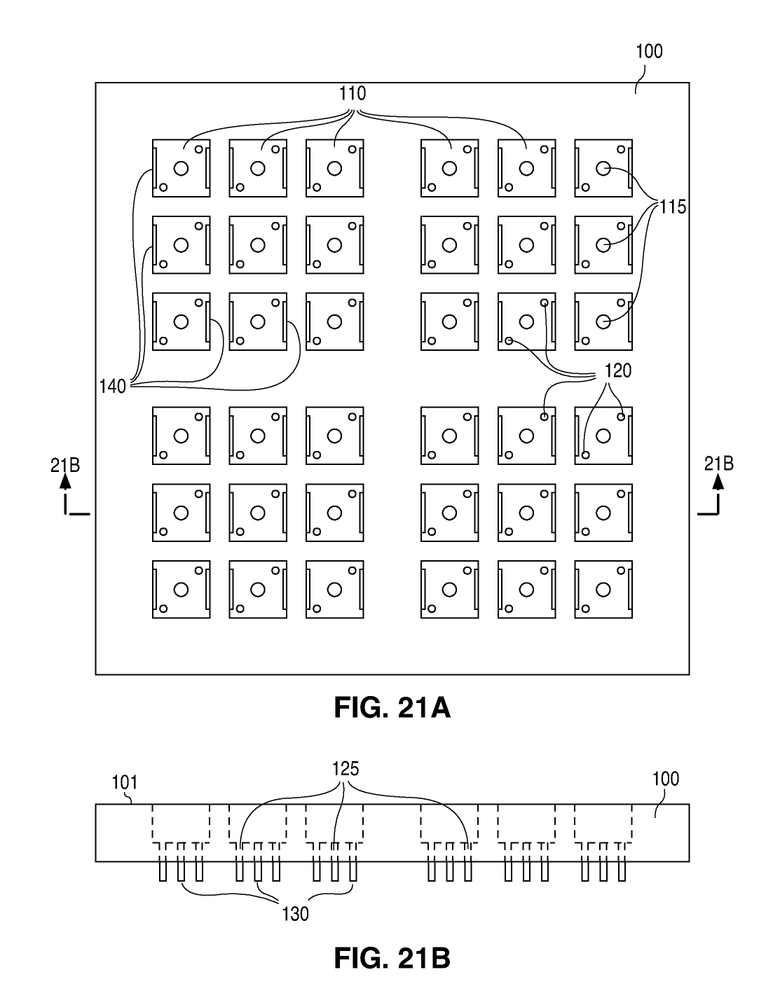

Another MEU embodiment of this invention is the device shown in FIGS. 21-23. There, base unit 100 is shown with an array of microfluidic chambers 110 impressed into the top surface 101 of base unit 100. As mentioned previously, “impressed” is merely meant to connote that the chambers are imbedded into base unit such that each chamber is below top surface 101 of base unit 100 and not to suggest any particular way in which the chambers are formed, which in fact can be by any means known or that might become known in the art. Each chamber is dimensioned so as to be capable of containing one and only one intact live eukaryotic cell. For example, in a presently preferred embodiment of this invention, the chambers are sized such that each chamber will contain one primary human T cell, such cells having diameters of about 7 μm to about 11 μm. Of course, other chamber sizes for other sized cells are within the scope of this invention. A presently preferred primary array of chambers comprises 6 chambers in a row with each row comprising a column of 6 chambers.

Each chamber of this invention comprises one or more ports in the floor of the chamber. A “port” is simply a lumen that extends from the top surface of a base unit to the bottom surface of the base unit such that the top and bottom surfaces are fluidically connected. An essentially centered port 115 is included as one of the ports and it is usually, although not necessarily, dedicated to the creation of a negative or a positive pressure at the outlet of the port into the chamber when the outlet is blocked by a cell. As used herein, a “negative pressure” refers to the withdrawal of air from the chamber such that, should the outlet of the port into the chamber be blocked by a cell, a slight vacuum would be created in the port to hold the cell in place in the chamber. The cells can then be subjected to a variety of fluidic conditions such as microporation buffers, mRNA in fluid carrier, reagents in solvents, wash solutions, etc. and the fluids could be removed from the chambers without the cells being flushed out of their individual chambers along with the fluids. As used herein, a “positive pressure” refers to a flow of air or other gas into a chamber through a port. If a cell is in the chamber, it having been held there by the above-described negative pressure, the stated positive pressure will be created by the inflowing air or other gas, which will push the cell out of the chamber for eventual collection. The use of the positive pressure is optional but may be useful, possibly even necessary, if cells become adhered to the bottom of a chamber.

Each chamber, in addition to its essentially centered port comprises one or more additional ports 120. These ports are used to introduce into and remove from the chambers various reagents such as, without limitation, electroporation buffers and mRNA-containing fluidic carriers.

The outlet 125 of each port into the chambers is separated from the chamber proper by a porous diffusion barrier which may be wire, gauze, polymeric or other material. The pores in the barrier are of sufficiently small size as to create a gentle, conformal inflow of whatever is being introduced into or withdrawn from the chamber so as to not deleteriously affect relatively fragile eukaryotic cells as they are being subjected to negative pressures, positive pressures and the introduction and removal of a potential multitude of fluidic media comprising biological and chemical reactants. A diffusion barrier comprising a gauze-like screen having pores about 1 μm in diameter is presently preferred.

The inlet 130 of each port, that is, that end of the ports at the bottom side of the base unit, is adapted for coupling to an external source of negative pressure, positive pressure and various fluidic reagents or reagent-containing fluids that may be used with the device of this invention. Any type of fitting known in the art and adaptable to the micro scale can be used such as, without limitation, simple force fittings, swage locks, luer locks and the like. The inlet of the ports may comprise the female fitting or the male fitting of the coupling device or some of the ports may be female fittings and some ports may constitute male fittings. Any combination of fittings and segments of fittings are within the scope of this invention.

Electrodes 140 are operatively coupled to the walls of the individual chambers. By “operatively coupled” is meant that the electrodes may be directly attached to the surface of the walls of a chamber or they may be separated from the surface by another entity such as, without limitation, an insulator or polymeric separator. The electrodes are set essentially opposite one another. That is, if a chamber is square or rectangular, the electrodes are placed on opposite walls of the chamber. It a chamber is round, the electrodes are placed essentially diametrically opposite one another. Other chamber shapes are of course possible and any and all shape variants are within the scope of this invention. As nearly as possible, however, it is presently preferred that the electrodes be placed opposite one another so as to be optimally situated for electroporation of a cell contained in the chamber.

As used herein, an “array” as applied to chambers of this invention refers to a plurality of two or more chambers. An array can be described by the number of chambers in a row and a number of chambers in a column. For example, without limitation, a 4×4 array describes an array with 4 chambers in each row and 4 chambers in a column beneath each chamber of the top row of chambers. The array would then constitute a total of 16 chambers. It is not necessary that the number of chambers in the rows of an array be the same as the number of chambers in a column. Thus, for example without limitation, arrays that are 3×2, 4×6, 8×9, etc. are entirely possible and are fully within the scope of this invention.

A presently preferred primary array, that is the array of all the chambers impressed into base 100, is 18×18 or a total of 324 chambers. Base 100 with its 324 chambers is referred to herein as a “first reaction tier.” In the first reaction tier, each chamber is a separate and distinct reaction vessel into which a single eukaryotic cell is placed. The cells are then subjected to a voltage to effect electroporation of the cells and subsequent introduction of non-integrating mRNA into each cell. Since the cells are completely fluidically isolated from one another, the mRNA introduced into each cell can be the same as or different from the mRNA introduced into each other cell. The mRNA medium can then be removed from the chambers and the modified cells can either be removed from the device, with like-transgene infected cells being collected together, or the transgene-infected cells can be further manipulated by introduction of a second reagent into each chamber. Again, the second reagent used in each chamber may be the same as or different from the second reagent used in each other chamber. If desired, the second reagent can then be removed and a third reagent, a fourth reagent and so on can be introduced into each cell in a similar manner. In general, for experimental reproducibility purposes, several chambers, usually contiguous in a de facto subarray, are treated the same. When all cells have been treated as desired, like-treated cells can be collected sequentially by releasing the negative pressure being applied to cells in chambers that have been similarly treated and collecting those cells, then releasing the negative pressure in second set of chambers with a second set of similarly treated cells, collecting those cells, and so on. During the collection process, if cells do not of their own accord float out of chambers when the negative pressure is removed, a positive pressure can be applied to gently push the cells out of the chambers.

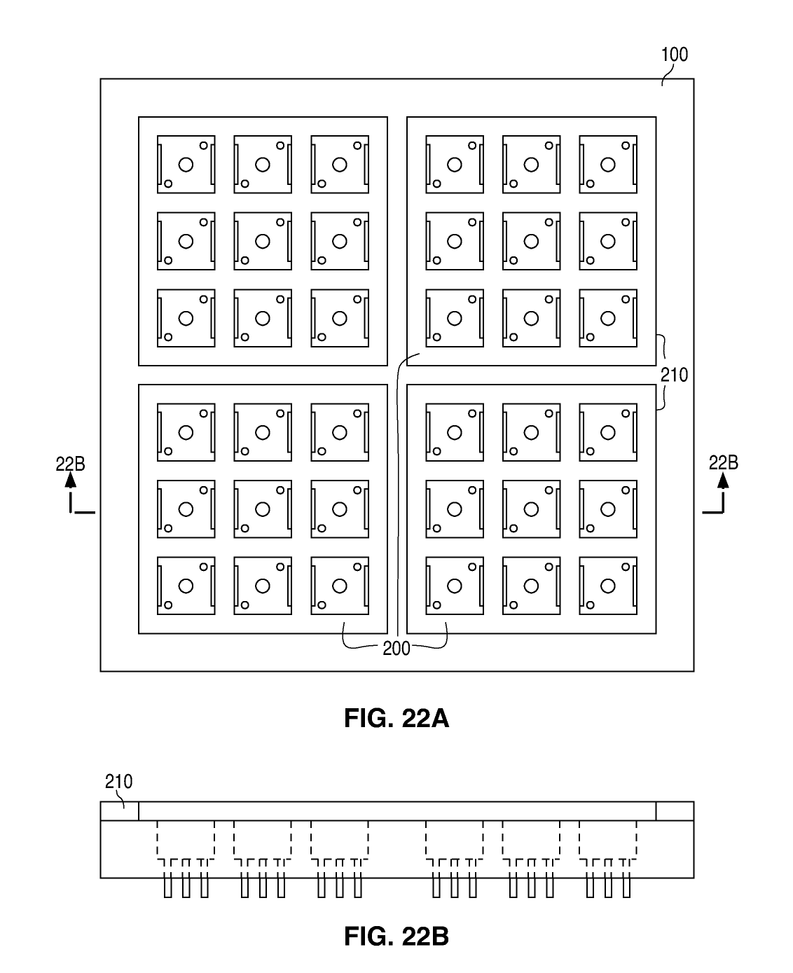

FIG. 22 shows a microfluidic device of this invention that is an extension of the device of FIG. 21. In FIG. 22, the full array of chambers in the FIG. 21 device is separated into subarrays 200 by walls 210 coupled to the top surface of base unit 100. This subarray of chambers is referred to herein as the “second reaction tier.” The walls are coupled in such a manner as to render each subarray 200 fluidically isolated from each other subarray 200. As is readily apparent from FIG. 22, the chambers in each subarray 15 would all be subject to contact with whatever reagent were to be placed in the volume created by walls 210. The height of walls 210 must be sufficient to prevent any fluid mixing between subarrays. That is, walls 210 can be any height with the proviso that the subarrays must be kept fluidically isolated from one another when reagents are introduced into each subarray volume. A wall 210 height about 10 times the height of the chamber walls is presently preferred, this height permitting the use of automatic precision microfluidic pumps to introduce and remove reagents from each subarray 200 volume. In this manner, as is presently preferred, first reaction tier and second reaction tier manipulation of cells can be carried out totally mechanically and, if desired, automatically. As with the first reaction tier, the same or different reagents may be introduced into each subarray volume of the second reaction tier including the serial introduction of multiple reagents into each subarray 115. Similarly treated cells can be collected in the same manner as mentioned above with regard to the first reaction tier. That is, release of negative pressure in chambers with similarly treated cells is followed by collection of those cells and so on. Also, as with the first reaction tier, if desired or necessary, the negative pressure can be replaced with a positive pressure to push similarly treated cells from their chambers.

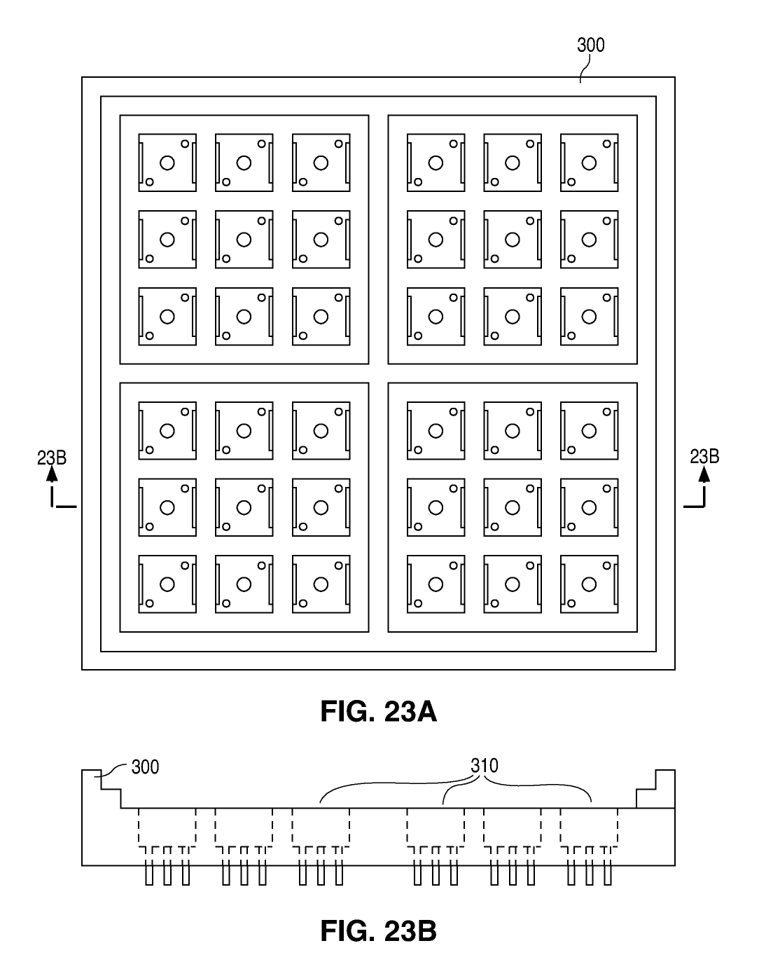

FIG. 23 shows a microfluidic device of this invention that is an extension of the device of FIG. 22. In FIG. 23, wall 300 is coupled to wall 200 such that all of chambers 110 are enclosed by yet another volume, this volume being referred to herein as a “third reaction tier” 310. Here, all of the cells in all of the chambers can be reacted with the same reagent or consecutive reagents.

It is, of course, entirely possible to use the entire three reaction tier device but effectively use only tier 1, only tier 2, only tier 3 or any 2-tier combination thereof. That is, once all cells have been electroporated and infused with mRNA, no other reaction may be carried out on in the individual chambers. Rather, a reagent or mixture of reagents or successive reagents or mixtures of reagents may be introduced into the second reaction tier. The manipulation of the cells may cease here and the variously treated cells collected or the third reaction tier may be used as described above. It is also possible to use the third reaction tier alone by first microporating the cells in the chambers and then proceeding directly to filling the volume created by the walls of the third reaction tier with a reagent, mixture of reagents or consecutive reagents of mixtures of reagents.

While the primary purpose of the above-described device is to first infect cells with non-integrating RNA and then to perform various experiments on such cells, it is to be understood that the same device can be used without microporation and simply be used to conduct various multifaceted experiments on cells. This would require simply not subjecting the cells in the chambers to an electroporating voltage. Such uses of the device herein are fully within the scope of this invention. Also, cells could be microporated and then treated with reagents other than non-integrating mRNA for other experimental purposes.

The method of using the MEUs of FIGS. 21, 22 and 23 is quite straight-forward. A plurality of eukaryotic cells of interest are generally suspended in an appropriate buffer, which will be known or relatively simply ascertained by those skilled in the art. A negative pressure is then applied to the chambers through one of the ports in the floor of the chamber, preferably at present the center port. Droplets of the suspension are then placed in or on each chamber and left there until a single cell has entered each chamber and has been entrapped therein. In the alternative, the suspension could be poured over the chambers as a whole and the device manipulated such as by tilting in all directions until, again, a single cell has been entrapped in each chamber. Then excess suspension is removed from the device leaving the cells in each chamber. The cells may optionally be washed to remove the suspension buffer. At this point, if electroporation is not to be an element of the particular experiment, the cells can treated with a variety of reagents, biological and/or chemical, with all chambers being treated the same of individual chambers or arrays of chambers being treated differently, i.e., with different reagents or different order of reagents. For the primary purpose of this invention, however, the next action would be to add an electroporation buffer found to be appropriate for the purpose to each chamber, either by simply pouring it over the surface of the device or by introduction into the chamber through one of the additional ports in the floor of the chambers. An appropriate voltage is then applied between the electrodes, again, the appropriate voltage either being know from the art or easily ascertainable by those skilled in the art. After a readily determined time interval for the cells to electroporate, a reagent is introduced into each chamber. For the purposes of this invention, the first reagent used is a non-integrating mRNA. The mRNA is placed in an appropriate fluid, known or readily ascertained by those skilled in the art and then introduced through one of the additional ports in the floor of the chambers using a precision microfluidic control system, likewise as such are known or may become known in the art. The same or different mRNAs may be introduced into all chambers, some individual chambers or some arrays of chambers. When the mRNA has been electro-transferred into the cells, the voltage is turned off at which time the voltage induced poration reverses. The electroporation buffer is removed and the mRNA-infected cells are washed with a appropriate buffer. At this time, the cells may be isolated or they may be subject to further treatment. If they are to be isolated and if some of the cells have been infected with different mRNAs, those infected the same can be isolated by removing the negative pressure in chambers containing similarly treated cells. The cells may then simply float out of the chambers of their own accord and can be collected. Optionally, a positive pressure can be applied most practically through the same port that was used to apply the negative pressure and the cells are gently pushed out of the chambers. By sequential removal of the negative pressure in chambers with similarly treated cells with the optional subsequent positive pressure, all similarly treated cells can be collected.

If further experimentation on groups of cells is desired, the second reaction tier can be used. In a presently preferred embodiment, the volume of the array of chambers of the second tier is also microfluidic so that the same microfluidic control system used in the first reaction tier can be used to fill the volume of the second reaction tier with the desired reagent. When it has been determined that the cells have reacted as planned, the same approach used to collect similarly treated cells as described above for transfected cells can be used.

If yet further treatment of the cells as a whole is desired, the third reaction tier can be used. This tier, in a presently preferred embodiment, is microfluidic, that is, it is amenable to addition of reagent-containing fluids manually using such devices, without limitation, syringes and micropipettes. After an appropriate time for the final reaction to occur has passed, the third reaction tier reactants are removed, the cells washed and then collected as described above.

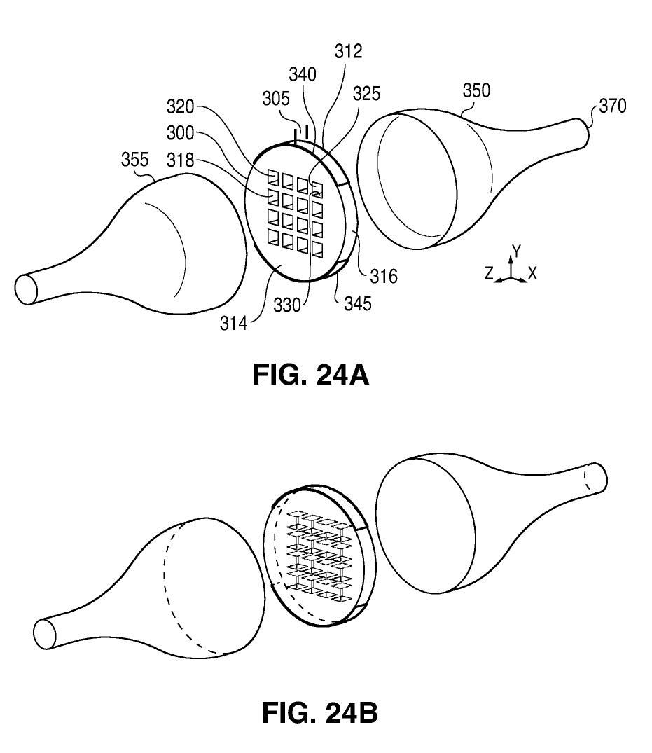

Yet another MEU embodiment of this invention is shown in FIG. 24. Whereas the devices of FIGS. 21, 22 and 23 are intended for experimental purposes, the MEU of FIG. 24, while it can certainly be used for purely experimental purposes and such use is clearly within the scope of this invention, is also intended for clinical applications. As one aspect of such use, the elements of the FIG. 24 device are intended to be simple, relatively inexpensive and individually replaceable for ease and economy of use.

The device of FIG. 24 first comprises an orifice plate 300. Orifice plate 300, which can be of any desired shape but most simply and preferred at present it is circularly shaped disk 310, has an inlet surface 312, an outlet surface 314, an outer edge 316 and one or more through-holes 320 sized such that one eukaryotic cell at a time can pass through each hole. The diameter of the disk is optional but preferably as small as possible given the constraint of the number of through-holes in orifice plate 300. The thickness 305 of the disk, which is the same as the wall surface 318 thickness of the through-holes is determined by the time necessary for cells passing through holes 320 to be electroporated and transfected with non-integrating mRNA. Since the time that a cell is in through-hole 320 is determined in part by the flow rate of the fluid in which the cells are suspended in through-hole 320, it is understood that such parameters can vary extensively and need not be expressly set forth herein. Those skilled in the art will be able to readily and without undue experimentation match the appropriate plate thickness 305 with an appropriate flow rate to permit cells to remain within holes 320 for the required period of time to effect electroporation and transfection. Each through-hole 320 has a positive electrode 325 and a negative electrode 330 operatively coupled to its wall 340. The electrodes are placed as nearly directly opposite one another as possible given the shape of through-holes 320, which may be any shape that permits cells to pass through. Since one presently preferred shape for through-holes 320 is square, electrodes 325 and 330 are place on opposite walls 340 and 345 of through-holes 320. Another presently preferred shape of through-holes 320 is circular, in which case electrodes 325 and 330 are placed diametrically opposite one another. Electrodes 325 and 330 are connected to positive electrode connection 340 and negative electrode connection 345, which are both operatively coupled to outer edge 316 of orifice plate 300 where they are available for connection to an external voltage source. An inlet exterior source adapter 350, through which unelectroporated cells enter the system on their way to the orifice plate, and an outlet adapter 355, through which electroporated cells exit the system after having passed through the orifice plate, are operatively coupled to the orifice place on opposite sides thereof. By “operatively coupled” is meant that the contact surfaces of the adapters and the orifice plate may simply be the surfaces of the adapters and the plate or there may be another substance, such as, without limitation, a sealing polymer, or another device such as, without limitation a gasket between the surfaces of the adapters and the orifice plate. Whatever the connections may comprise, the connections themselves must be fluid-tight, that is, must not allow for ingress or egress of anything that is flowing through the orifice plate. The connection between the two adapters and the orifice plate, when all are in place and a fluid-tight seal has been made, is such that positive electrode connector 340 and negative electrode connector 345 on outer edge 316 of orifice plate 300 are accessible for connection to an external voltage source. The end of the inlet adapter opposite the end that is coupled to the orifice plate is operatively coupled to two or more external source connection ports 370. External source connection ports 370 are operatively coupled to sources of substances to be used in the device. If only two ports are provided, one connection port is connected to a cell source and the other port is connected to a source of a nucleic acid with which the cells are to be non-integratedly transformed. If more than two ports are used, the other ports may be connected to sources of other biological or chemical reagent with which it is intended that the electroporated cells are to be treated in addition to the nucleic acid. At present, the nucleic acid is a non-integrating mRNA, while the additional substance, if any, can be whatever else the operator wishes to treat the cells with. The treated cells pass through the orifice plate, are electroporated and transfected and, if desired, further manipulated in an additional selected manner, and then exit the system through the outlet adapter into a collection device, which may be, without limitation, a flask, a bottle, a cuvette and the like.

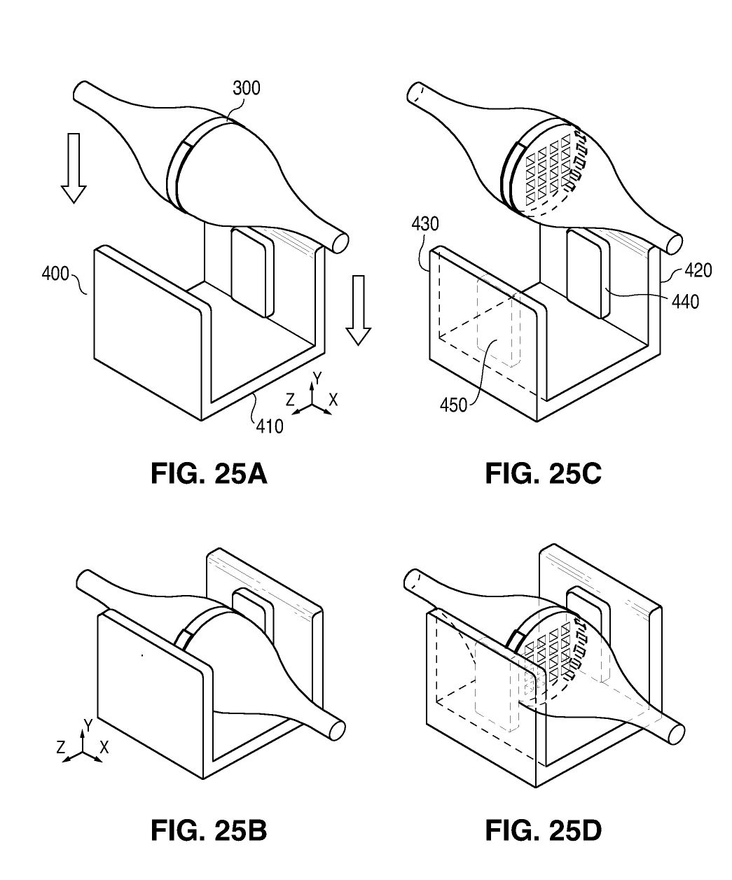

Voltages are provided to the through-hole electrodes by means of U-shaped device holder 400 shown in FIG. 25. U-shaped device holder 400 is comprised of base 410 and parallel side walls 420 and 430. Side wall 420 is operatively coupled to positive pole electrical contact 440 and side wall 430 is operatively coupled to negative pole electrical contact 450. Walls 420 and 430 are spaced apart such that, when orifice plate 300 is placed between them, positive electrode connection 340 is electrically coupled to positive pole electrical contact 440 and negative electrical connection 445 is electrically coupled to negative pole electrical contact 450. Positive pole electrical contact 440 and negative pole electrical contract 450 are coupled to an external voltage source, not shown.

By inclusion of an optional cell separator between a cell source and the inlet adapter, it is possible to use the MEU device of FIG. 24 in a clinical treatment mode. The system is assembled under sterile conditions. One sterile external course connection port is operatively coupled to sterile source of nucleic acids. Another external source port is operatively coupled to a sterile syringe needle, that is, a needle with a central lumen extending its entire length as such are well known in the art. The sterile needle is inserted into a blood vessel of a subject, which may be any living organism having blood vessels, but is preferably a mammal and most preferably at present a human being. The blood of the subject is thus the source of external cells, the desired cells being separated from the blood in the cell separator with the desired cells continuing on into the MEU device and the rest of the blood components being returned to the subject. The desired cells are presently preferred to be primary human T-cells when the subject is a human being. The chosen cells and the selected nucleic acid, preferably at present non-integrating mRNA, then pass through the orifice plate wherein the cells are electroporated and transfected with the mRNA. Those cells then pass out of the MEU through the outlet port, which has another sterile syringe needle at its end away from the device, which needle has been inserted into another blood vessel of the subject. In this manner it is possible to provide a constant source of transfected cells to a subject in need thereof.

A method of treating a disease in a subject comprises, for example without limitation, the following procedure. A subject or patient (the terms are used interchangeably herein) who is afflicted with a disease known to be, found to be or suspected to be amendable to treatment using transfected cells is identified. The patient is hooked up to the device of this invention by means of a syringe needle that has been inserted into a blood vessel. Blood is withdrawn from the patient and optionally sent to a cell separator where cells intended to be electroporated and electro-transfected are separated from the other blood components. The selected cells can be any mentioned anywhere in this document or any others that it might be found are useful as transfected cells in the treatment of any disease. Presently preferred cells are primary human T-cells. The remaining blood components are returned to the patient while the separated cells are then introduced into the through-holes of the orifice plate of the device of FIG. 25 in which the electrodes on the walls of the through-holes have been activated; i.e. a voltage has been applied across the space between the electrodes. A separate source of a nucleic acid, which like the cells may be any nucleic acid found to be of value for the treatment of a disease, is simultaneously passed through the through-holes such that as the cells pass through they are electroporated and the nucleic acid can ingress into the cells through the created pores. A number or representative useful nucleic acids are mentioned elsewhere herein but a presently preferred nucleic acid is mRNA. After the cells have been electro-transfected, they pass through the outlet of the device, through a conventional i.v.-type tubing to a syringe needle that has been inserted into a blood vessel of the patient, and thence into the patient. In this manner, a constant source of non-integratedly transfected cells can be continuously provided to a patient for as long as the treating medical practitioner deems necessary.

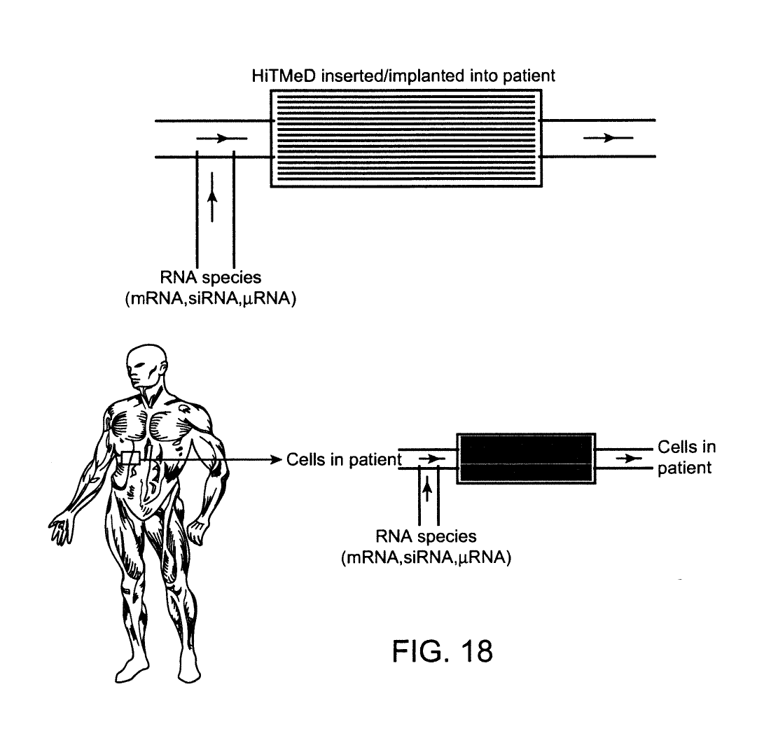

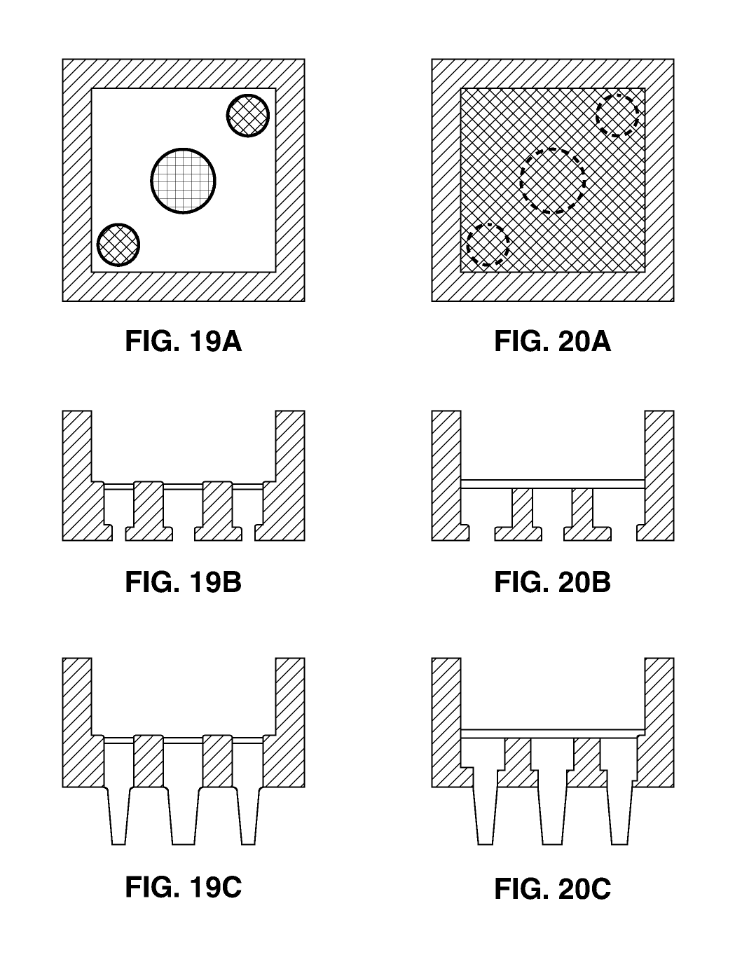

The overall size of some of the devices of this invention that are indicated to have clinical utility will depend on the size of the various components and the housing containing some or all of them. It is, however, envisioned that the components and the housing will be sized so as to be implantable in the body of a patient as shown in FIG. 18. Micro scale versions of many of the components of the devices herein, other than the novel MEUs of this invention, are either available or will be achievable by those skilled in the art based on the disclosures herein.

As used herein, “genetic material” refers to DNA and RNA that, when inserted into a living cell, expresses or leads to the expression of a desired protein regardless of whether the genetic material is actually integrated into the organism's genome or simply inserts into the nucleus and makes use of the replication machinery therein to express the protein.

In one aspect the present invention relates to a microfluidic electroporation device and method of use for efficient, reproducible, continuous insertion of genetic material, fluorochromes (tags) and/or proteins into cells by electroporation. For example, without limitation, an integrated system that is capable of high throughput electroporation of a large number of clinical grade cells in parallel fashion is an aspect of this invention. The process may be carried out in numerous ways including, without limitation, using individual component devices with manual transfer of the product of one component into the next component, to rendering the entire process, from obtaining the desired cell type for transfection to the delivering the transfected cells to a subject in need thereof, in a totally closed system. Further, it is contemplated that all of the components may be miniaturized such that the entire closed system can be implanted in the body of the subject for continuous long-term therapy. The closed systems, whether macro or micro scale, can mimic the operating condition provided by a GMP facility or one that operates under standard blood banking protocols. Thus, what the devices of the current invention in effect offer is a “GMP-in-a-box” that will facilitate the transfer of integrating and non-integrating genes and other nucleic acids into cells under standard blood-banking and good manufacturing practices as established by the FDA and AABB (American Association of Blood Banks). That is, cells can be recursively collected from a subject, for example without limitation, by venipuncture or apheresis, a nucleic acid coding for a desired protein can be transferred into the cells or into a desired subset of cells such as, without limitation, T and NK cells, and the modified cells can be re-infused into the patient to effect treatment, all in a sterile closed system that can be operated in a clinical setting. Advantages of this process compared to those currently in use in gene therapy and non-integrating cell therapy include, without limitation, the adoptive transfer of minimally manipulated cells at a cost substantially below that of ex vivo culturing and an inherent improvement in the biologic functioning of the modified cells since cell differentiation, which accompanies propagation needed to achieve clinically-meaningful numbers of cells is not required. That is, the devices of the current invention can be coupled with high throughput so as to allow patients receiving gene transfer therapy to receive back large numbers of cells within hours of collection followed by gene transfer. This constitutes a fundamental shift in the way gene therapy is perceived.

In sum, until now, the introduction and expression of transgenes has required major investments in research, development, manufacturing and regulatory support. While this has resulted in the development of state-of-the-art GMP facilities that are capable of executing complex manufacturing processes, the technology is expensive and time consuming. Due primarily to the expense involved, just a few patients around the world are currently or ever will be able to benefit from gene therapy or its closely allied technique, non-integrating cell transfection therapy. The ability to operate the current invention in a clinical setting means that gene therapy will be available to a many more patients of diverse economic means than is even imaginable using current technologies including, significantly, patients in under-developed and developing nations.

The devices and methods described herein will be amenable to a variety of applications, e.g., gene therapy for the prevention and cure of inheritable or inherited diseases, and both gene therapy and transient transfection treatment of diseases known to be, or become known to be or that are suspected of being susceptible to treatment by such cell-based therapy. A particularly notable disease for which transient transfection may be useful is cancer.

A device of the present invention can not only introduce desired genetic material into cells but also can monitor the cell's response. This can be accomplished by providing a marker that is co-expressed along with the desired genetic material by transfected cells and which can be detected by various means to identify cells that in fact have been transfected. While numerous such marker techniques are known to those skilled in the art, and all are within the contemplation of this invention, one non-limiting example of such is use of a fluorescent tag that can be detected by a fluorescence detector.

The efficiency of the device and method of the present invention lends itself readily to adoptive transfer of minimally manipulated cells, with reduction in costs associated with extensive ex vivo culturing. Improvements in the biologic functioning of the genetically modified cells are through use of the present invention also very beneficial, since cell differentiation, which accompanies the cell propagation needed to achieve clinically-meaningful numbers of T and NK cells, can be avoided.

The present invention can improve the efficiency of the transfer of genetic material into immune-derived cells for the treatment of cancer using novel cell electroporation and gene material delivery techniques.

Non-viral gene transfer has been used to introduce DNA plasmids expressing desired transgenes into cells. Currently, non-viral gene transfer uses commercially available technology to achieve ex vivo electrotransfer of RNA and DNA in cells in cuvettes. This method of gene transfer, however, is inefficient due to low transfection and integration efficiency and is not readily amenable to GMP processes due to difficulties in engineering a closed system to accomplish the transfer.

To address the above problem, the present invention provides microfluidic genetic material transfer devices which can be operated within most blood banking centers in developed and developing nations, thereby significantly broadening the distribution of gene therapy technology. These devices can be coupled with high throughput so as to allow patients receiving genetic material therapy to reiteratively receive back large numbers of cells within hours of collection and modification using the method of this invention, resulting in a fundamental shift in the way such therapy is perceived and delivered.

An aspect of this present invention is a multi-stream channel comprising parallel lanes. The multi-stream channels can allow cells and buffer solutions to flow through while maintaining their respective streamlines due to low Reynolds numbers for the respective streams resulting in laminar flow. The multi-stream channel can further include a plurality of electrodes in a pattern that generates multiple electroporation zones in the channel. The electroporation zones can include mechanisms to control the duration and electric voltage of electroporation so as to control the number and size of pores on a cell flowing through the channel. The size of pores can range from about 10 nm to about 500 μm. An array of multistream channels are also within the contemplation of this invention to provide a high throughput device capable of producing therapeutically significant quantities of transfected cells in a relatively short period of time.

In an aspect of this invention, a method of genetic material therapy is provided that comprises: identifying a patient suffering from a disease; selecting a cell-type for treatment of the disease; removing a fluid containing cells of the selected cell-type from the patient's body; separating the cells from other constituents of the fluid; optionally activating the separated cells; electroporating the separated cells; contacting the electroporated cells with one or more therapeutic DNAs and/or RNAs to form non-integrated DNA- and/or RNA-containing cells; optionally evaluating the DNA- or RNA-containing cells for conformance with release criteria; returning the DNA- and/or RNA-containing cells into the patient's body; and, repeating the removing, separating, optionally activating, electroporating, contacting, optionally evaluating and returning as necessary to treat the disease.

As used herein, a “source of living cells” refers to any source known to those skilled in the art. Examples include, but are not limited to, commercial sources of specific cell types or mixtures thereof, whole blood either taken from a subject and transferred to a storage container for later use in the methods herein, or taken from a subject and transferred directly to a device of this invention.

If a source, such as whole blood, that contains a mixture of many cell types is used it may be desirable to separate out the cells of interest using a “cell selection component.” If cell selection is opted for, any means known to those skilled in the art may be employed. These include, without limitation, centrifugation techniques, i.e., density-based techniques such as apheresis, magnetic techniques employing antibodies to tag specific cell types with small magnetic particles that are later isolated, and use of tetrameric antibody complexes (TACs) to remove unwanted cells from the selected cell type, etc.

The cell-type can be any type of cell known or found to be useful for a particular therapeutic purpose. That is, cells such as, without limitation, T cells, NK cells, dendritic cells (or antigen presenting cells), B cells, monocytes, reticulocytes, fibroblasts, hematopoietic stem/progenitor cell, mesenchymal stem cells, other stem cells, tumor cells, umbilical cord blood-derived cells and peripheral-blood derived cells may be used.

The cell-type can be numerically expanded and/or cultured ex vivo prior to insertion of the nucleic acid.

The DNA and/or RNA can code for therapeutic agents including, without limitation, an enzyme, a chimeric antigen receptor, a hormone, an antibody, a clotting factor, a notch ligand, a recombinant antigen for vaccine, a cytokine, a cytokine receptor, a co-stimulatory molecule, a T-cell receptor, FoxP3, a chemokine, a chemokine receptor, a luminescent probe, a fluorescent probe, a reporter probe for positron emission tomography, a KIR deactivator, hemoglobin, Fc receptors, CD24, BTLA, somatostatin, a transposase, a transposon for Sleeping Beauty or piggyback and combinations of any of the foregoing. The RNA can be chemically modified to improve persistence. Further, the RNA can be prepared in vitro from a DNA plasmid which has been modified (e.g. a polyA tail can be added and/or untranslated region from beta-globin can be included) to confer improved persistence of the RNA species (Holtkamp et al., Blood, (2006) 108:4009-17). The RNA can be any of mRNA, siRNA and microRNA or combinations thereof. If desired, the RNA species can be combined with DNA species, such as the electrotransfer of mRNA transposase from, for example without limitation, Sleeping Beauty (Wilber et al., Mol. Ther. (2006) 13:625-30) or piggyBac (Wilson et al., Mol. Ther. (2007) 15:139-45.)) and a DNA plasmid transposon such as that coding for, without limitation, a chimeric antigen receptor.

The above procedures can be carried out in a variety of ways. Preferably at present, all steps are performed in a closed, sterile, unbreached recirculating system that provides (i) providing a source of living cells, (ii) optionally selecting certain cells from the source, (iii) optionally focusing the selected cells, (iv) optionally activating the selected cells, (v) mixing the cells with DNA and/or RNA, (vi) electroporating the cells, (vii) optionally detecting transfected cells, and then (viii) collecting the transfected cells. For example, providing a source of living cells can be accomplished by, without limitation, venipuncture, apheresis, use of an in-dwelling central catheter, or use of a central intravenous catheter. Selecting one or more cell types can also comprise, without limitation, apheresis. Cells may also be obtained by biopsy or surgery. Activating the cells can be accomplished by treating the cells with a substance that causes the cells to undertake a particular function. For example without limitation, T and NK cells are known to become cytotoxic when activated by exposure to cytokines, such as IL-2, or growth factors. Electroporating cells can comprise using a Nucleofector® system (Lonza Köln AG, Germany). Contacting the electroporated cells with one or more therapeutic DNA(s) and/or RNA(s) can comprise contacting the cells with a fluid containing the therapeutic DNA(s) and/or RNA(s). Electroporation and contacting the electroporated cells with a fluid containing the therapeutic DNA(s) and/or RNA(s) can be performed substantially simultaneously. That is, the cells can be mixed with the DNA and/or RNA prior to subjecting the cells to electroporation. Returning the therapeutic DNA- and/or RNA-containing cells can comprise the same route by which the cells were provided in the first place, i.e., venipuncture, an in-dwelling central catheter, a central intravenous catheter, etc., or it may be accomplished using a canulating lymphatic system.

Any disease known to be, or that may become known to be in the future, or that is suspected of being, amenable to gene therapy can be treated using the methods and devices of this invention. Cancer, for instance, is presently known to be such a disease. Thus, a genetic material transfer therapy for cancer using the methods and devices of this invention might comprise removing a fluid containing T-cells and/or NK cells by apheresis, separating the T-cells and/or NK cells using a microfluidic cell separator, activating the cells by contacting them with IL-2, and then electroporating them using Nucleofector®. Contacting the electroporated T-cells and/or NK cells with therapeutic DNA and/or RNA can comprise contacting them with mRNA coding for a CD19-specific chimeric antigen receptor. Electroporation and contact with the mRNA coding for CD19-specific chimeric antigen receptor can be conducted substantially simultaneously. The CD19-specific chimeric antigen receptor can comprise CD19RCd28.

As used herein, a “subject” refers to any living entity that might benefit from treatment using the devices and methods herein. As used herein “subject” and “patient” are used interchangeably. A subject or patient refers in particular to a mammal such as, without limitation, cat, dog, horse, cow, sheep, rabbit and preferably at present, a human being that may be an adult patient or a pediatric patient.

### Electroporation

As previously mentioned herein, electroporation is a well-established method for delivery of drugs and genes into cells. The basic concept of electroporation is that controlled application of an electric field to a mammalian cell membrane can temporarily increase membrane permeability as a result of the formation of nano-scale pores in the membrane. The use of microfluidic devices for cell electroporation is, however, novel and offers several advantages compared to current electroporation methods. For instance, microelectronic patterning techniques can reduce the distance between the electrodes in the microchips such that low voltages can be used to generate high electric field strengths. Cell handling and manipulation should also be easier since the channels and electrodes can be comparable in size to cells. Cell electroporation, separation and detection can be integrated on a single platform. Transformation efficiency can be improved. A micro-electroporation device may be integrated with other devices in a complex analyzer. Such advanced integration will be possible because cellular manipulations in the present invention are performed in simple flow systems.

As shown in Example 1, a chimeric antigen receptor (CAR) can be successfully introduced into cells by electroporation and thereafter expressed by the cells.

### Recirculating Closed System

As noted previously, an aspect of the present invention is a recirculating closed system for recursively extracting cells from a patient, electroporating them, transiently transfecting RNA or DNA into them and then returning them to the patient where expression of the transfected gene provides the desired therapeutic result. The recirculating closed system can include:

a fluid removal component having a proximal and a distal end and a lumen extending from the proximal to the distal end wherein the proximal end of the fluid removal component is inserted into a vein of the patient;

a first tube having a proximal and a distal end, the proximal end of which is coupled to the distal end of the fluid removal component;

a cell separation device having an inlet and an outlet wherein the distal end of the first tube is coupled to the inlet of the cell separation component;

a second tube having a proximal and a distal end, the proximal end of which is coupled to the outlet of the cell separation component;

an electroporation component having an inlet and an outlet wherein the distal end of the second tube is coupled to the inlet of the electrophoresis component;

a third tube having a proximal and a distal end, the proximal end of which is coupled to the outlet of the electroporation component and the distal end of which is coupled to a proximal end of a fluid return component, a distal end of which is inserted into a vein of the patient.

In an aspect of this invention, the cell separation component and the electroporation component can be directly coupled to one another; that is, there is no second tube.

Likewise, the fluid removal component and the fluid return component can be one and the same. For example, without limitation, the fluid removal component and the fluid return component can comprise a single needle or an in-dwelling central intravenous catheter.

The recirculating closed system can comprise a single channel design that can electroporate single cells in a flow-through manner. An illustrative schematic of a channel design is shown in FIG. 5, where cells and buffer solutions flow in alternating lanes of a multi-stream channel. Because of the low Reynolds number, viscous forces predominate over inertial forces, laminar flow ensues and there is no pronounced convective mixing of the solutions. Thus, the fluids in each lane can maintain their respective streamlines and can be directed down the channel with mixing of solutes occurring only due to the relatively slow process of diffusion. Low Reynolds number flows can be used to focus a solution of cells into a single stream of cells.

Electrodes can be patterned into the channels using any suitable technique such as microlithography. Multiple electroporation zones can be created to control transfection efficiency. For example, a single cell may travel through multiple sets of electrodes before being transfected, thereby introducing multiple electroporation zones, as illustrated in FIG. 2, which can increase the probability of transfection and thus overall transfection efficiency, and one or a plurality of cross-channels can be used to introduce desired reagents, RNA, and/or media to the electroporated cells. Other factors include electric field strength for electroporating the cells and the rate of fluid flow which can be controlled so that cells are exposed to electric fields for a desired amount of time.

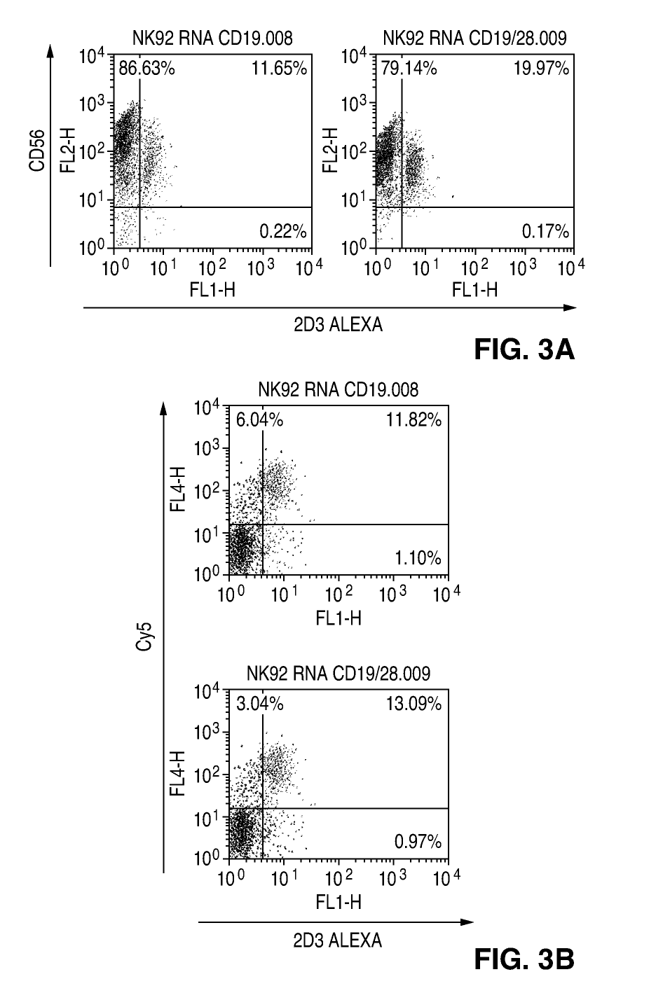

The recirculating closed system can incorporate a detection device to measure the efficiency of the system. For example, fluorescence labeling technology can be used to determine the efficiency of the system. Such a detection scheme can include an optical detection method that uses a membrane-impermeable fluorescent stain to monitor cellular membrane integrity (Yeh et al., J. Immunol. Methods (1981) 43:269-75, Schmidt et al. Cytometry (1992) 13:204-08). In addition, by transfecting electroporated cells with fluorescently-labeled target RNA and then measuring intracellular fluorescence, not only how many cells were successfully electroporated but also how many tagged RNA molecules were transfected into the cells can be monitored. FIG. 3 shows a fluorescence labeling technology using cuvettes. This method permits evaluation of electrical parameters, voltage and pulse length needed for optimal cell membrane permeabilization. Further, whether compounds expected to stabilize membrane pores and thereby improve transfection efficiency are in fact doing so can be examined.

Microfluidic devices of this invention can be used to separate T and NK cells from other cells in the blood to avoid electroporation of the other cells. The T and NK cells can then be directed to channels which have an orifice plate which focuses the electric field and allows for single-cell electroporation with high efficiency. The electric field can be tailored by the orifice plate, allowing control of the magnitude and localization of the transmembrane voltage. Since electroporation of a cell results in a resistance change of the membrane, membrane permeation can be detected by characteristic ‘jumps’ in current that correspond to drops in cell resistance. The microfluidics device can, of course, be a high throughput device.

A plurality of channels can be created on a microfluidic device described herein according to the above procedures. For example, an array of channels can be created each of which can be used for single cell electroporation. Arrays of electrodes can likewise be created to perform multiple electroporation operations, which can last for minutes, hours, days or even months, preferably at present from about twelve to about twenty-four hours.

The microfluidic device can include disposable parts or components such as, for example, disposable microfluidic electrotransfer cassettes to avoid cross-contamination.

### Method of Use

The method and system described herein have a variety of applications. For example, the system and method can be used for recursive electrotransfer of DNA and/or RNA species, e.g., mRNA to enforce transgene expression, siRNA to down regulate disease causing gene expression, and microRNA to regulate transgene expression for integrating and non-integrating gene transfer. The transgenes can be used to express a protein or peptide in a cell or an organism using the method described herein, which include, but are not limited to, transgenes expressing chimeric antigen receptors (including humanized sequences); hormones, e.g., insulin; antibodies; clotting factors, e.g., hemophilia factors; Notch ligand; recombinant antigens for vaccines; cytokines; cytokine receptors; proteins or peptides expressed by imaging transgenes (e.g., thymidine kinase, iodine simporter, somatostatin receptor); co-stimulatory molecules; T-cell receptors; FoxP3; chemokines; chemokine receptors, e.g., CXCR4; luminescent probes; fluorescent probes; genes to de-activate KIR; hemoglobin; Fc Receptors; CD24; BTLA; or somatostatin.

The transgenes can be expressed in human and non-human cells including, but not limited to: T cells; NK cells; B cells; monocytes; red blood cells (reticulocytes); stem cells, e.g., hematopoietic stem cells, mesenchymal stem cells; tumor cells; umbilical cord blood-derived cells; peripheral-blood derived cells; or cells that have undergone ex vivo numerical expansion.

### Method of Clinical Trials

The efficient introduction and expression of desired transgenes into viable immune cells such as T cells makes possible a new class of clinical trials based on the recursive infusion of genetically modified cells. This can have major advantages over current trial design as it (i) does not require integrating transgenes and can avoid the need for oversight by National Institutes of Health Office of Biotechnology Activities (NOH OBA) with associated stringent regulatory oversight and down-stream long term follow up expenses, (ii) avoids the need for production of expensive and potentially hazardous vectors (such as retrovirus or lentivirus) for transfection of immune cells, (iii) allows genetically modified cells to be available on demand, and (iv) uses a minimally-manipulated cell product which maintains in vivo viability (thereby avoiding replicative senescence associated with extensive ex vivo propagation), and avoids in-depth and expensive release testing.

For example, the microfluidic device described herein can be used to assess the efficacy of recursive adoptive transfer of autologous CD19-specific T cells in patients with chemo-refractory (lethal) B-lineage acute lymphoblastic leukemia. An inter-patient dose escalation can evaluate feasibility of giving 1 to 7 doses of 10⁹/m² CD19-specific T cells over a two-week period. Correlative studies can establish persistence of infused cells based on imaging technologies (e.g., PET imaging) and excretion of beta-HCG as well as determine the potential for an immune response against infused T cells.

The release/in-process testing for the infusion of CD19-specific genetically manipulated T cells are summarized in Table 1 below. These tests can be modified by an ordinary artisan to suit the application and transgene expression desired.

TABLE 1
Summary of “Release” assay and “In Process” testing

| Test | Release Criteria | In-Process Tests | Test Method |
|---|---|---|---|
| Sterility | Negative for | | Gram and KOH |
| | bacteria and fungi | | stains |
| Sterility | | Negative for bacteria | U.S.P. |
| | | at 14 days; Negative | |
| | | for fungi at 28 days | |
| Mycoplasma | | Negative for | PCR |
| assay | | mycoplasma | |
| Endotoxin | <5 EIU/Kg recipient | | Chromogenic LAL |
| assay | body weight/hour of | | |
| | T-cell infusion | | |
| Chimeric: ζ | | 75-kDa Protein Band | Western Blot with |
| receptor | | | human CD3ζ-specific |
| expression | | | primary Ab |
| Cell surface | ≧90% CD3⁺ and ≧10% | | Flow cytometric |
| phenotype | Transgene⁺ | | evaluation |
| Viability | ≧60% Viable | | Trypan blue |
| | | | exclusion test |
| CD19-specific | ≧30% Specific | | 1 hr non-radioactive |
| cytolytic | lysis at 50:1 (E:T) | | lysis assay |
| activity | against a CD19⁺ | | |
| (potency) | cell line | | |

Non-integrative plasmid (NIP) technology, shown in Example 2, can be used to ensure that the genetically modified infused cells will (i) numerically proliferate and survive in vivo (ii) express the transgene appropriately and (iii) home to the disease site (e.g., tumor site). For example, two transgenes can be used to monitor the persistence/biodistribution of the genetically modified cells in vivo.

The transgene can be tagged with beta-HCG (human choriogonadotrophic hormone), the secretion of which can be used as a measure of gene transfer and beta-HCG excretion in urine, can be used as a measure of in vivo survival of infused genetically modified cells. This information in turn will provide for a measurement of tumor killing vis-a-vis the persistence of the infused cells.

In cancer patients, interaction of somatostatin receptor with ¹¹¹-IN-Octreotide (OctreoScan™, Hazelwood Mo.) can be used to monitor the progress of treatment. This somatostatin receptor with OctreoScan™ has been exploited to image many tumors. Somatostatin receptor scintigraphy is highly sensitive for tumor detection especially for unsuspected lymph node metastasis. Somatostatin receptor scintigraphy have detected tumors which were not detected by MRI or CT. OctreoScan™ is readily available and FDA approved for tumor imaging in patients. Thus, one can tag a somastatin receptor to a desired transgene, electroporate the cells, transfect the transgene and evaluate the interaction of the OctreoScan™ in vitro prior to assessing function of the transgene and to correlate function with clinical outcome.

Immune-based therapies based on transient gene transfer to cells (e.g., to T and NK cells) have a variety of applications. Non-viral gene transfer can be used to introduce RNA and DNA to deliver transgenes to achieve personalized medicine using cost-effective technology which can be broadly implemented.

The method described herein can be used as a therapeutic measure in the field of pediatric oncology. For example, a pediatric patient can undergo apheresis and reinfusion of genetically modified cells the same day using blood banking practices already in place. This can allow the development of investigator-initiated pediatric oncology drugs/therapeutics based on the patient's immune system, leading to multi-institution gene therapy treatments recruiting large numbers of patients, leading to a portable genetic modification system at low cost and applicable to the application of genetically modified immune cells for multiple classes of neoplasms and pathogens.

### EXAMPLES

### Example 1

### Electroporation of mRNA to T-cells

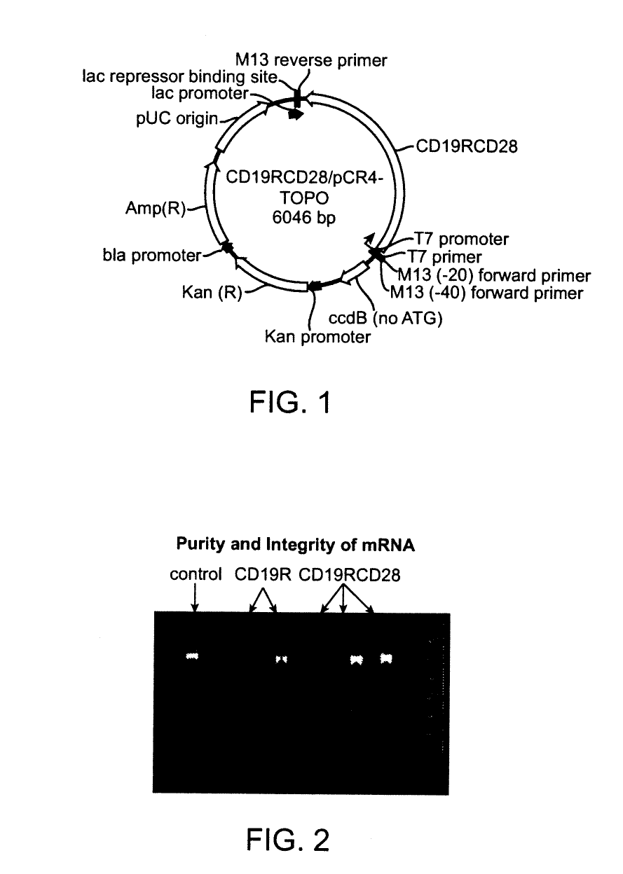

In this example, CD19-specific chimeric antigen receptor (CAR) was used as the transgene to be expressed in T cells. To evaluate the electroporation of desired mRNA, and whether electroporated mRNA can be expressed in primary cells and in cell lines, a T7 promoter was generated based on vectors containing second generation CAR designated CD19RCD28 (FIG. 1). Integrity of these vectors was determined by standard molecular biology methods. To generate mRNA specific for CD19R and CD19RCD28 from this vector, the DNA vectors were linearized (FIG. 2A) and the mRNAs were prepared using an MEGAscript kit (Ambion, Tx) according to manufacturer instructions. Purity and integrity of mRNAs were determined by gel electrophoresis (FIG. 2B). Purified RNAs were then electroporated into a Jurkat T-cell line, a NK92 cell line and primary NK cells using Amaxa Biosystems Nucleofector™ II and the expression of CD19R and CD19RCD28 were determined by FACS analysis (FIG. 3A).

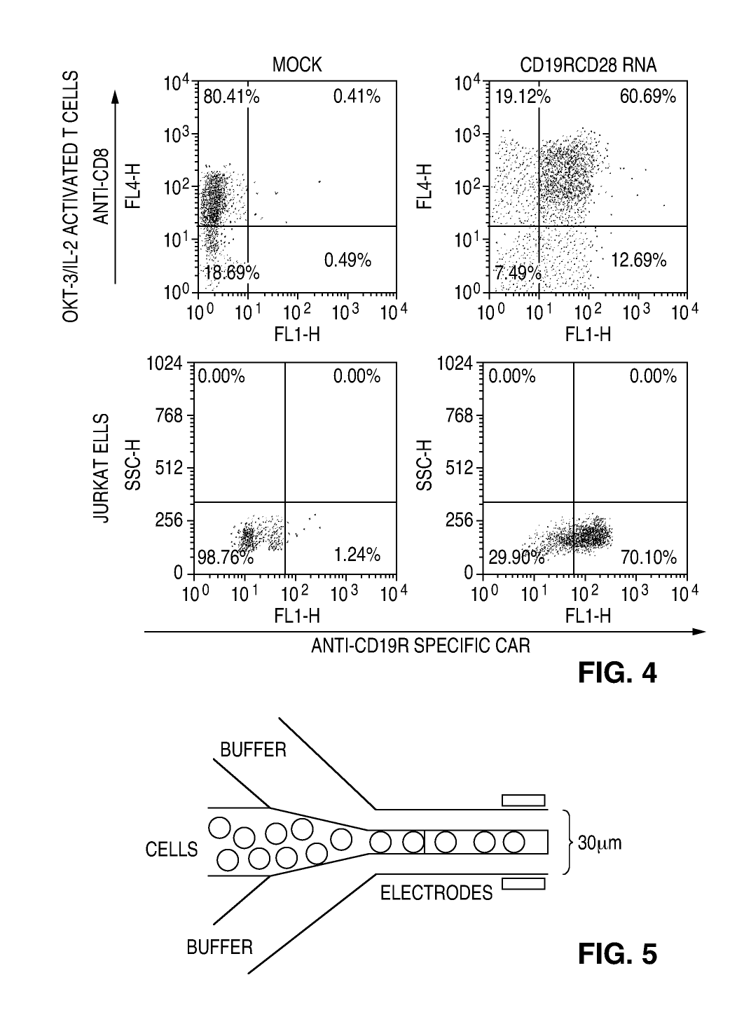

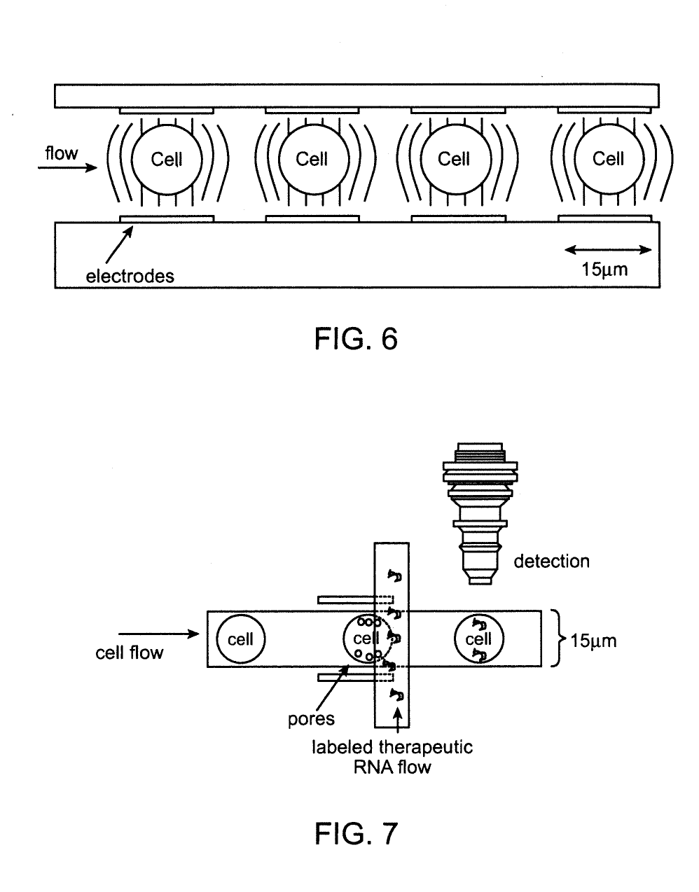

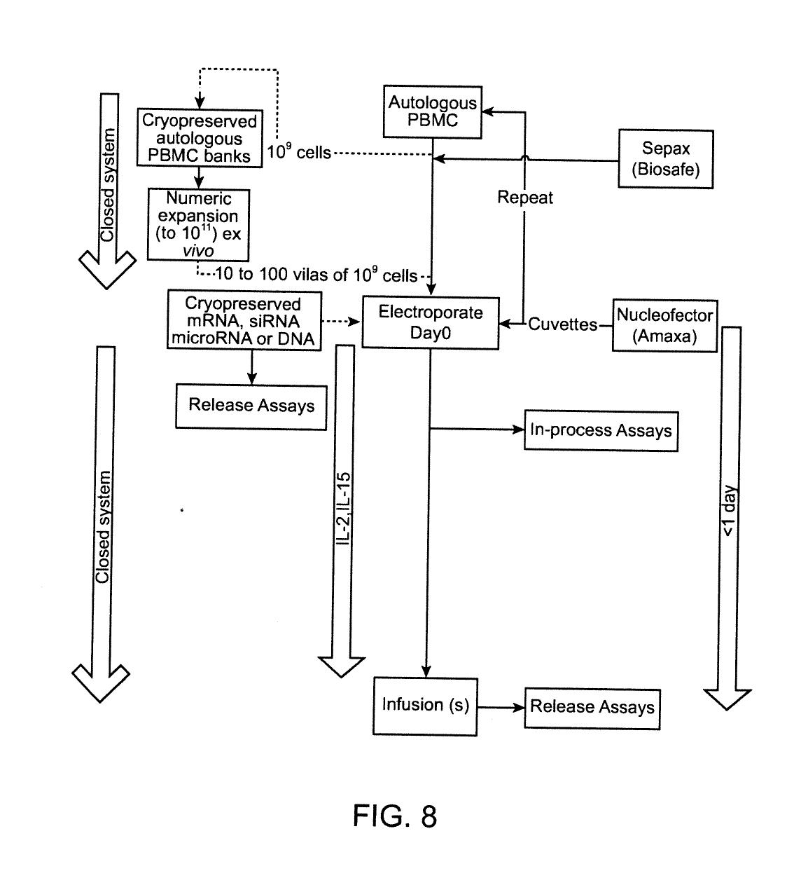

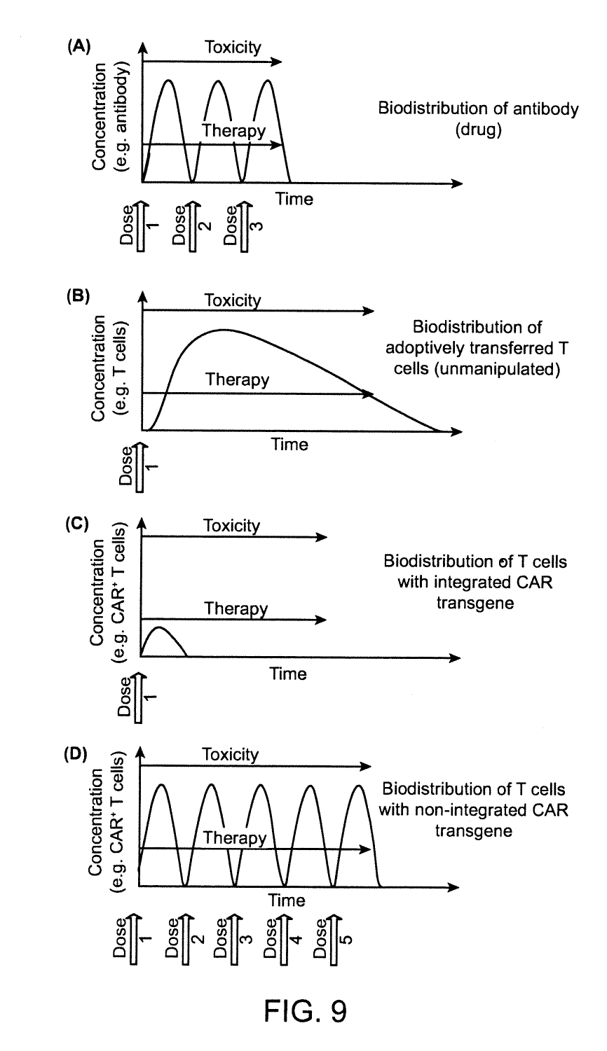

As seen in FIG. 3A, when the NK 92 cell line was electroporated with CD19RCD28, 20% of the cells were positive for 2D3-Alexa labeled CD19. In contrast however, the primary NK cells were negative for CD19R. These data demonstrate that electroporation conditions for primary NK cells would be different then NK cell lines. RNA electroporation in the Jurkat T-cell line was also successful, with 10% of the cells positive for CD19R. When Cy5 labeled CD19R was electroporated into the cells and FACS analysis performed to determine the presence of mRNA (FIG. 3B), the labeled mRNA could be detected in NK92 and Jurkat cells for up to 24 hrs (FIG. 4).

### Example 2

### Non-Integrated Plasmid (NIP) Study

Anti-CD20-IL-2 ICK was demonstrated to bind specifically to CD20⁺ tumors as well as IL-2R⁺ T cells and infusing a combination of anti-CD20-IL-2 ICK with CD19R⁺ T cells improves in vivo T-cell persistence leading to an augmented clearance of CD20⁺CD19⁺ tumor, beyond that achieved by delivery of the ICK or T cells alone.

### Plasmid Expression Vectors

The plasmid vector CD19R/ffLucHyTK-pMG co-expresses the CD19R chimeric immunoreceptor gene and the tripartite fusion gene ffLacHyTK (22). Truncated CD19, lacking the cytoplasmic domain (Mahmoud M S, et al., Blood (1999) 94:3551-8), was expressed in ffLucHyTK-pMG to generate the plasmid tCD19/ffLucHyTK-pMG to co-express the CD19 and ffLucHyTK transgenes. Bifunctional hRLucZeo fusion gene that co-expresses Renilla koellikeri (Sea Pansy) luciferase hRLuc and zeomycin-resistance gene (Zeo) was cloned from the plasmid pMOD-LucSh (InvivoGen, San Diego, Calif.) into peDNA3.1⁺ (Invitrogen, Carlsbad, Calif.), to create the plasmid hRLuc:Zeocin-pcDNA3.1. Propagation of cell Lines and primary human T cells

Daudi, ARH-77, Raji, SUP-B15, K562, cells were obtained from ATCC (Manassas, Va.) and Granta-519 cells from DSMZ (Braunschweig, Germany). An EBV-transformed lymphoblastoid cell line (LCL) was kindly provided by Drs. Phillip Greenberg and Stanley Riddell (Fred Hutchinson Cancer Research Center, Seattle, Wash.). These cells were maintained in tissue culture as described (Serrano L M, et al., Blood (2006) 107:2643-52). IL-2Rβ⁺ TF-1β cells were kindly provided by Dr. Paul M. Sondel, (University of Wisconsin, Madison, Wis.) (Farner N L, et al., Blood (1995) 86:4568-78). Human T-cell lines were derived from UCB mononuclear cells after informed consent and cultured as previously described (Cooper L J, et al., Blood (2003) 101:1637-44; Riddell S R, Greenberg P D, J Immunol Methods 1990; 128:189-201).

### Immunocytokines

The anti-CD20-IL-2 (DI-Leu16-IL-2) ICK was derived from a de-immunized anti-CD20 murine mAb (Leu16). Anti-GD₂-IL-2 (14.18-IL-2) which recognizes GD₂ disialoganglioside served as a control ICK with irrelevant specificity for a B-lineage tumor line used in this study (EMD Lexigen Research Center, Billerica Mass.) (Gillies S D, et al., Proc Nad Acad Sci USA 1992; 89:1428-32).

### Non-viral Gene Transfer of DNA Plasmid Vectors

OKT3-activated UCB-derived T cells were genetically modified by electroporation with CD19R/ffLucHyTK-pMG (Serrano LM, et al., Blood (2006) 107:2643-52). ARH-77 was electroporated with hRLuc:Zeocin-pcDNA3.1 using the Multiporator device (250V/40 μsec, Eppendorf, Hamburg, Germany) and propagated in cytocidal concentration (0.2 mg/mL) of zeocin (InvivoGen).

### Flow Cytometry

Fluorescein isothiocyanate (FITC), or phycoerythrin (PE), conjugated reagents were obtained from BD Biosciences (San Jose, Calif.): anti-TCRαβ, anti-CD3, anti-CD4, anti-CD8, anti-CD25, and anti-CD122. F(ab′)₂ fragment of FITC-conjugated goat anti-human Fcγ, (Jackson Immunoresearch, West Grove, Pa.) was used at 1/20 dilution to detect cell-surface expression of CD19R transgene. Leul6 and anti-CD20-IL-2 ICK (100 μg each) were conjugated to Alexa Fluor 647 (Molecular Probes, Eugene Oreg.). Data acquisition was on a FACS Calibur (BD Biosciences) using CellQuest version 3.3 (BD Biosciences) and analysis was undertaken using FCS Express version 3.00.007 (Thornhill, Ontario, Canada).

### Chromium Release Assay

The cytolytic activity of T-cells was determined by 4-hour chromium release assay (CRA). CD19 specific T cells were incubated with 5×10³ chromium labeled target cells in a V-bottom 96-well plate (Costar, Cambridge, Mass.). The percentage of specific cytolysis was calculated from the release of ⁵¹Cr using a TopCount NXT (PerkinElmer Life and Analytical Sciences, Inc, Boston, Mass.). Data are reported as mean±SD.

### Immunofluorescence Microscopy

CD19R⁺ T cells (10⁶) and CD19⁺CD20⁺tumor cells (10⁶) were centrifuged at 200 g for 1 min and incubated at 37° C. for 30 minutes. After gentle re-suspension, the cells were sedimented, supernatant was removed, and the pellet was fixed for 20 min with 3% parafomaldehyde in PBS on ice. After washing, the fixed T cell-tumor cell conjugates were incubated for 30 minutes at 4° C. with anti-CD3-FITC or Alexa Fluor 647-conjugated anti-CD20-IL-2 ICK. Nuclei were counterstained with Hoechst 33342 (Molecular Probes. Eugene, Oreg.) (0.1 μg/mL). Cells were examined on a Zeiss LSM 510 META NLO Axiovert 200 M inverted microscope. Hoechst 33342 was excited at 750 nm using Coherent Ti:Sapphire multiphoton laser, Alexa Fluor 647 at 633 nm using Helium-Neon laser, and FITC at 488 nm using Argon ion laser. Images were acquired with a Zeiss plan-neofluar 20×/0.5 air lens or plan neofluar 40×/1.3 NA oil immersion lens and fields of view were then examined using Zeiss LSM Image Browser Version 3,5,0,223 (configuration at cityofhope-org/LMC/LSMmett.asp).

### Persistence of Adoptively Transferred T Cells

Prior to the initiation of the experiment, 6-10 week old female NOD/scid (NODILtSz-Prkdcscid/J) mice (Jackson Laboratory, Bar Harbor, Me.) were γ-irradiated to 2.5 Gy using an external ¹³⁷Cs-source (JL Shepherd Mark I Irradiator, San Fernando, Calif.) and maintained under pathogen-free conditions at COH Animal Resources Center. On day −7 the mice were injected in the peritoneum with 2×10⁶ hRLuc⁺CD19⁺CD20⁺ARH-77 cells. Tumor engraftment was evaluated by biophotonic imaging and mice with progressively growing tumors were segregated into four treatment groups to receive 10⁷ CD19-specific T-cells (day 0) either alone or in combination with 75,000 U/injection (equivalent to ˜25 μg ICK(25)) IL-2 (Chiron, Emeryville, Calif.), 5 μg/injection anti-CD20—

IL-2 ICK (DI-Leu16-IL-2) or 5 μg/injection anti-GD₂-IL-2 ICK, given by additional separate intraperitoneal injections. Animal experiments were approved by COH institutional committees.

### In Vivo Efficacy of Combination Immunotherapies

Six to ten week old γ-irradiated NOD/scid mice were injected with 2×10⁶ hRLuc⁺ CD19⁺CD20⁺ARH-77 cells in the peritoneum. Sustained tumor engraftment was documented within 7 days of injection by biophotonic imaging. Mice in the four treatment groups received combinations of CD19-specific T cells (10⁷ cells in the peritoneum on day 0), anti-CD20-IL-2 ICK or anti-GD₂-IL-2 ICK (5 μg/injection in the peritoneum).

### Biophotonic Imaging

Anaesthetized mice were imaged using a Xenogen IVIS 100 series system as previously described (Cooper L J, et al., Blood (2005) 105:16221-31). Briefly, each animal was serially imaged in an anterior-posterior orientation at the same relative time point after 100 μL (0.068 mg/mouse) of freshly diluted Enduren™ Live Cell Substrate (Promega, Madison, Wis.), or 150 μL (4.29 mg/mouse) of freshly thawed D-luciferin potassium salt (Xenogen, Alameda, Calif.) solution injection. Photons were quantified using the software program “Living Image” (Xenogen). Statistical analysis of the photon flux at the end of the experiment was accomplished by comparing area under the curve using two-sided Wilcoxon rank sum test. Biologic T-cell half life was calculated as A=I×(½)^{(t/h)}(A=flux at time t, I=day 0 flux, h=rate of decay).

### Redirecting T Cells Specificity for CD19

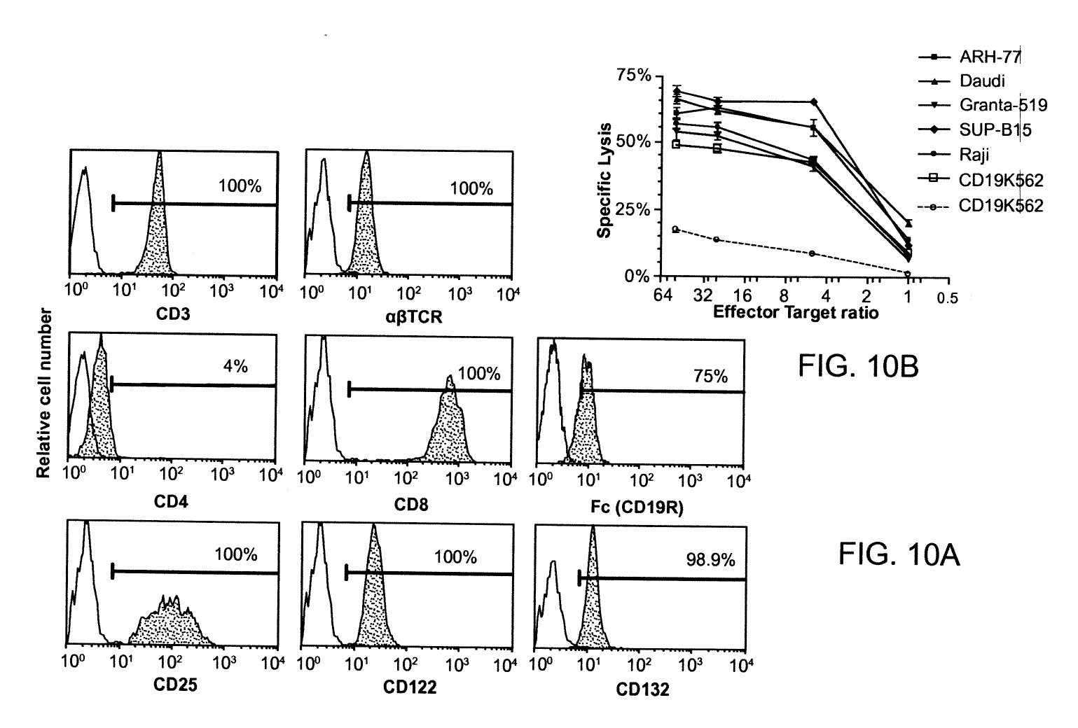

The genetic modification of UCB-derived T cells to render them specific for CD19 was accomplished by non-viral electrotransfer of a DNA expression plasmid designated CD19R/ffLucHyTK-pMG, that codes for the CD19R transgene (Cooper L J, et al., Blood (2003) 101:1637-44) and a recombinant multi-function fusion gene that combines firefly luciferase (ffLuc), hygromycin phosphotransferase and herpes virus thymidine kinase (HyTK) (Lupton S D, et al., Mol. Cell Biol. (1991) 11:3374-8), permitting in vitro selection of CD19R⁺ T cells with cytocidal concentration of hygromycin B and in vivo imaging after infusion of D-luciferin. Genetically modified ex vivo expanded T cells were CD8⁺; expressed components of the high-affinity IL-2 receptor (IL-2R) and CD19R transgene, as detected using a Fc-specific antibody (FIG. 10A). CD19R⁺ T cells could specifically lyse leukemia and lymphoma targets expressing CD19 with ˜50-70% of CD19⁺ tumor cells killed at an E:T ratio of 50:1 in a 4 hour CRA (FIG. 10B). The variability of lysis of the various B-cell lines could be attributed to the expression of various cell surface markers particularly the adhesion molecules (Cooper L J, et al., Blood (2003) 101:1637-44). Specific lysis of CD19⁺ K562 compared to CD19^{neg} K562 cells demonstrated that the killing of CD19⁺ tumor targets occurred through the chimeric immunoreceptor.

### Binding of Anti-CD20-IL-2 ICK

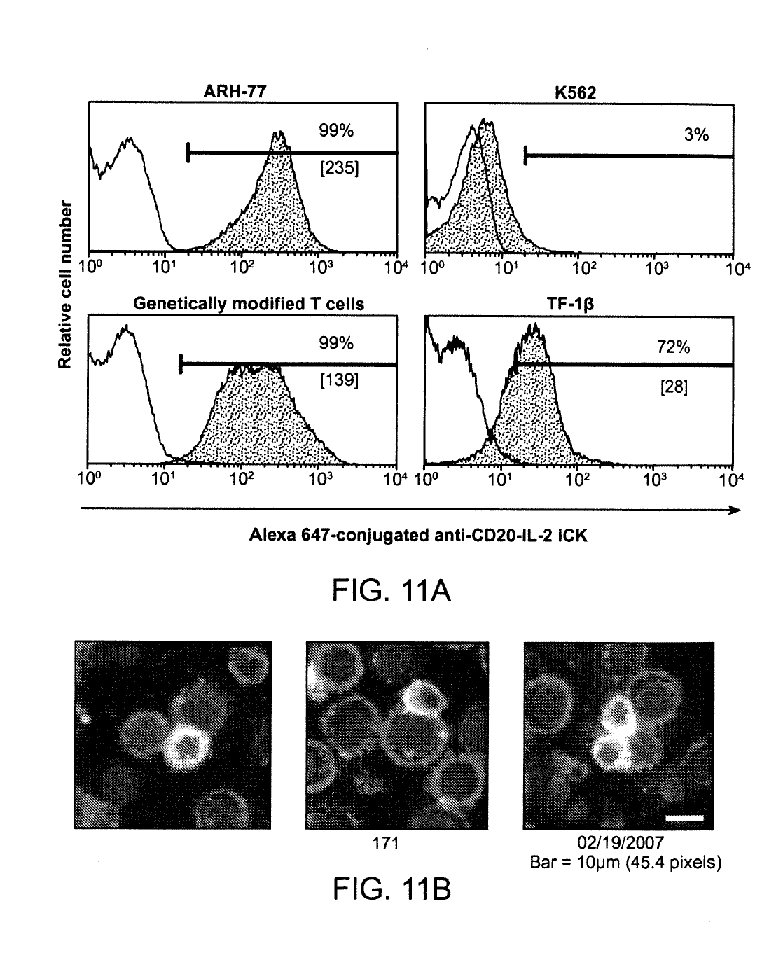

The ability of the anti-CD20-IL-2 ICK to bind to both B-lineage tumors and T cells was examined using flow cytometry and confocal microscopy. This ICK bound to CD20⁺ ARH-77 but not CD20^{neg} SUP-B15 and K562 cells, consistent with recognition of parental Leu16 mAb for CD20 (FIG. 11A) (Rentsch B., et al., Eur. J. Haematol. (1991) 47:204-12). The anti-CD20-IL-2 ICK, but not parental Leul6 mAb, bound to CD25⁺ genetically modified T cells and to TF-1 β, a tumor cell line genetically modified to express CD122 (IL-2Rβ) (Farner N L, et al., Blood (1995) 86:4568-78), which is consistent with binding of chimeric IL-2 via the IL-2R (FIG. 11A). The greater median fluorescent intensity (MFI) on T cells, compared with TF-1β, is consistent with binding of the ICK to the high-affinity IL-2R. Immunofluorescence confocal microscopy was performed to evaluate the localization of ICK on conjugates of CD19-specific T cells and CD20⁺ tumors. The confocal micrographs demonstrated cell-surface labeling of conjugates of tumor and T cells with Alexa Fluor 647-conjugated anti-CD20-IL-2 ICK (red) and T cells labeled with FITC-conjugated anti-CD3 (green). Areas of overlapping binding between deposition of ICK and anti-CD3 is depicted by a yellow color (FIG. 11B). These results show that T cells exhibit co-localization of CD3 and ICK on their surface initially but as they form a synapse with the tumor cell there seems to be a rearrangement of IL2R on the T cells towards the synapse leading to the presence of yellow signal extending well outside the synapse and leaving a green pocket opposite the synapse. The Alexa Fluor 647-conjugated parental anti-CD20 Leu16 mAb, lacking the chimeric IL-2 domain, binds CD20⁺ tumors, but not the genetically modified T cells (data not shown). In aggregate these data show that anti-CD20-IL-2 ICK can bind to CD20 molecules on B-lineage tumors and IL-2R on T cells and furthermore that this ICK can be deposited at the interface between tumor and T cells.

In Vivo T-Cell Persistence Given in Combination with ICK

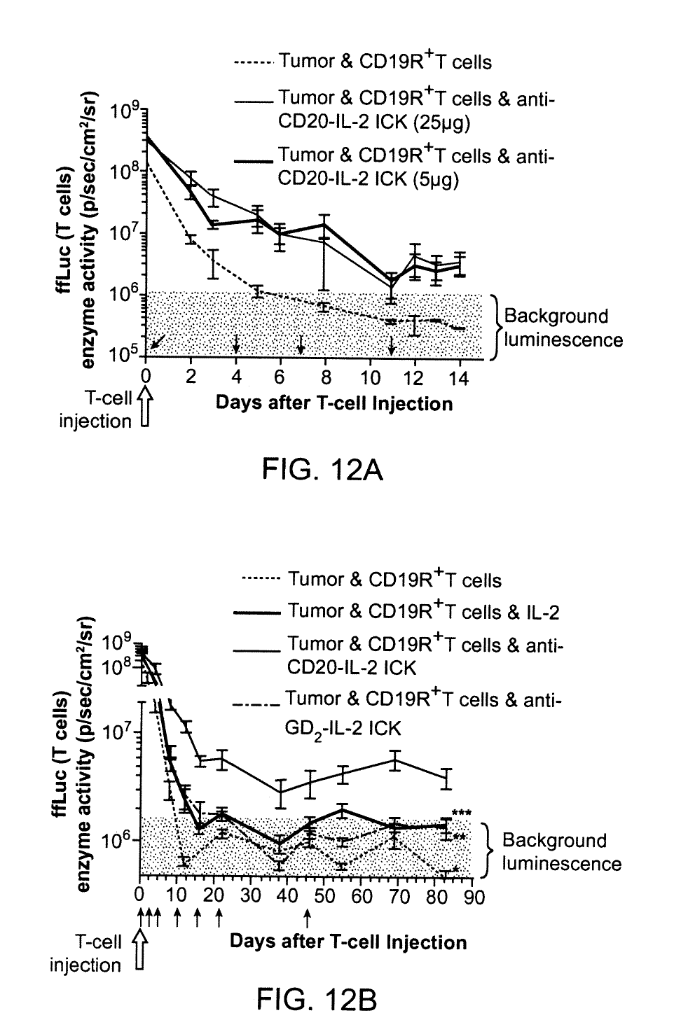

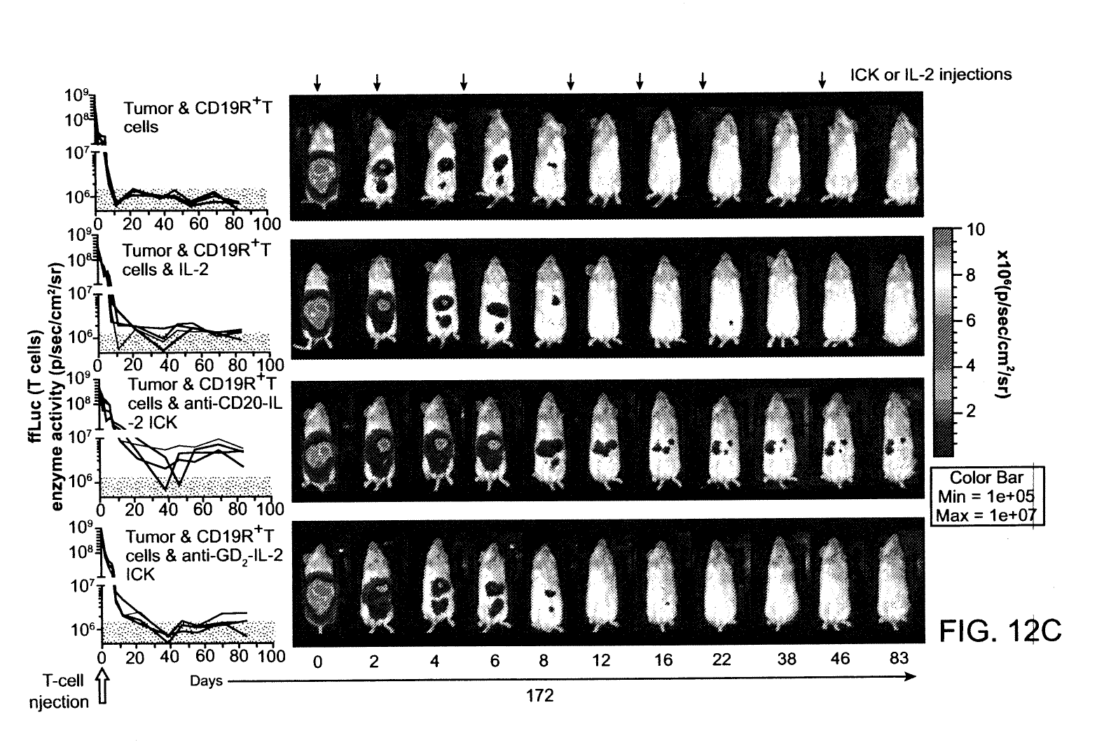

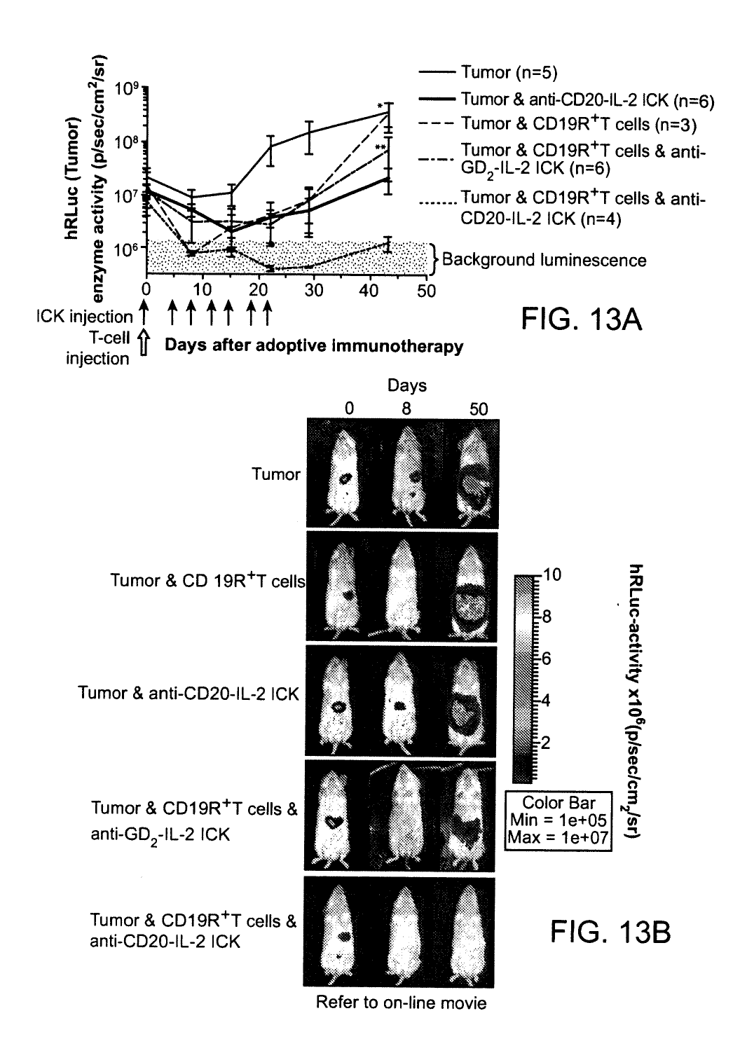

Having determined that the anti-CD20-IL-2 ICK could bind to tumor and T cells, whether infusions of anti-CD20-IL-2 ICK can improve the in vivo persistence of adoptively transferred genetically modified CD8⁺ T cells was evaluated. To achieve sustained loco-regional depositions of the anti-CD20-IL-2 ICK, the tumor line ARH-77 was chosen as a target for immunotherapy, since this is relatively resistant to killing by anti-CD20-specific mAb (Treon S P, et al., J. Immunother. (2001) 24:26371), and these results were confirmed in vivo in NOD/scid mice using Rituximab®. Initially, a dose of ICK was established that could both improve the in vivo survival of CD8⁺CD19R⁺ffLuc⁺ T cells, compared with adoptive immunotherapy in the absence of ICK, and not statistically alter tumor growth as monotherapy (FIG. 13). It was demonstrated that an ICK dose of both 5 and 25 μg can improve the persistence of infused T cells resulting in a T-cell ffLuc-derived signal detectable above background luminescence measurements (≦10⁶ p/sec/cm²/sr) 14 days after adoptive immunotherapy (FIG. 12A). Biologic half life of the infused T cells was determined by calculating the rate of T-cell decay (ftLuc activity) at the end of the experiment and expressed as the number of days required by the cells to achieve half the initial (Day 0) flux. Indeed, the biological half-life of the infused T cells was twice as long in mice that received ICK (1.09 d) compared with T cells given alone (0.43 d). As a further indication that infusion of the ICK may enhance the survival of adoptively transferred T cells, an approximately 300% (3-fold) increase was observed in the ffLuc-derived signal (day 12) as compared to day 11 when the ICK was injected in both the groups. As the relative in vivo T-cell persistence was similar for both of the ICK doses (p=0.86), 5 μg per ICK injection was used for subsequent experiments, a dose equivalent to ˜15,000 units of human recombinant IL-2 (Gillies S D, et al., Blood 2005; 105:3972-8).

To determine if the improved T-cell persistence was due to the binding of the ICK in the ARH-77 tumor microenvironment, a control ICK (anti-GD₂-IL-2 ICK) which does not bind to GD₂^{neg} ARH-77 was used. Furthermore, the ability of the anti-CD20-IL-2 ICK to potentiate T-cell survival was compared with administration of exogenous recombinant human IL-2. Longitudinal measurement of ffLuc-derived flux revealed that the infused T cells persisted longer in mice that received anti-CD20-IL-2 ICK, as compared to the untreated (p=0.01), IL-2-treated (p=0.02) and control ICK-treated (p=0.05) groups (FIG. 12B, 12C); the biological half lives of T cells in the groups being 1.7, 0.5, 1.0 and 0.7 days respectively. There was a difference (p<0.05) in the in vivo persistence of T cells accompanied by IL-2, compared with T cells given without this cytokine, which is consistent with the dependence of these T cells to receive T-cell help in the form of exogenous IL-2 to survive in vivo. No apparent difference was observed in the persistence (p=0.5) or biologic half-life (p=0.2) of adoptively transferred T cells between the mice receiving exogenous IL-2 or control ICK. These data support the hypothesis that the loco-regional deposition of the anti-CD20-IL-2 ICK at the CD19⁺CD20⁺ tumor site significantly augments in vivo persistence of CD8⁺ CD 19-specific T cells.

In Vivo Efficacy of ICK in Combination with CD19-Specific T Cell to Treat Established B-Lineage Tumor

In vivo investigation was performed to determine whether the ICK-mediated improved persistence of genetically modified CD19-specific T cells could lead to augmented clearance of established CD19⁺CD20⁺ tumor. A dose of T cells (10⁷ cells) was selected since this dose by itself does not control long-term tumor growth (FIG. 13). CD19-specific CD8⁺ T cells were adoptively transferred into groups of mice bearing established CD19⁺CD20⁺hRLuc⁺ ARH-77 tumor along with anti-CD20-IL-2 ICK, or control anti-GD₂-IL-2 ICK. Tumor growth was serially monitored by in vivo bioluminescent imaging (BLI) of ARH-77 tumor-derived hRLuc enzyme activity. Mice that received both CD19-specific T cells and anti-CD20-IL-2 ICK experienced a reduction in tumor growth with 75% of mice obtaining complete remission, as measured by BLI, at the end of the experiment (50 days after adoptive immunotherapy) (FIG. 13). It was found that the combination therapy of CD19R⁺ T cells and anti-CD20-IL-2 ICK was effective in reducing tumor growth as compared to no immunotherapy (p=0.01) and T cells given with an equivalent dosing of the control ICK (p=0.03). Even though the tumor burden seems to be increasing in the treated group, no visible tumor as seen by hRLuc signal was observed at the end of the experiment, as the flux remained below background level, consistent with a complete anti-tumor response. Mouse groups receiving T cells alone or T cells with control ICK showed a similar pattern of tumor growth, with an initial reduction around day 8, followed by relapse. All mice in the control group, which received no immunotherapy, experienced sustained tumor growth. Similar tumor growth kinetics were observed in mice that did or did not receive anti-CD20-O-IL-2 ICK in the absence of T cells (p>0.05 through day 50) and this is presumably a reflection of the dose regimen chosen for the ICK in this experiment. Increased doses of T cells or anti-CD20-IL-2 ICK delivered as monotherapies results in a sustained anti-tumor effect, but using these doses would preclude the ability to measure the ability of the ICK to potentiate T-cell persistence and improve tumor killing.

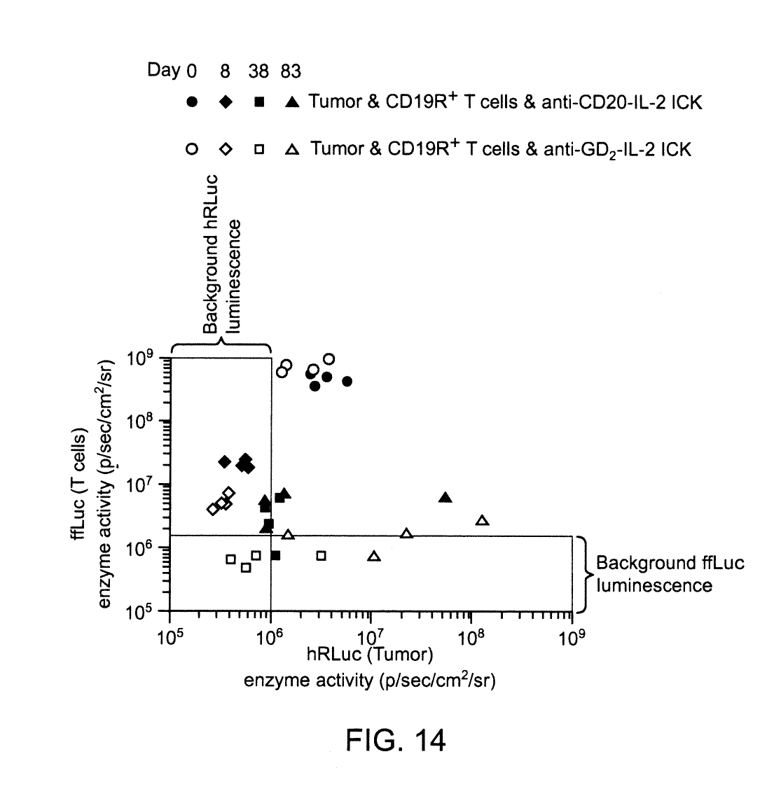

The ability to measure both ffLuc and hRLuc enzyme activities in the same mice allowed the determination of whether the persistence of adoptively transferred T cells directly correlated with tumor size for individual mice. This was accomplished by plotting ffLuc-derived T-cell flux versus hRLuc-derived tumor-cell flux from FIG. 12. Both groups of mice, which received CD19-specific T cells along with anti-CD20-IL-2 ICK/anti-GD2-IL-2 ICK, showed a drop in tumor burden at day 8, which is due to the T cells infused. However, the highest numbers of T cells (ffLuc activity; mean flux 4.7×10⁶ vs 1.5×10⁶ p/sec/cm²/sr) and lowest tumor burden (hRLuc activity; mean flux 1.4×10⁷ vs 4×10⁷ p/sec/cm²/sr) by day 83 (FIG. 14) was observed in the group receiving CD20-ICK, when compared to the control ICK-treated group. This analysis demonstrates that half the mice achieve an anti-tumor response (absence of detectable hRLuc activity) after combination immunotherapy with CD19R⁺ T cells and anti-CD20-IL-2 ICK. It was noted that there was continued T-cell persistence (ffLuc activity) in the anti-CD20-IL-2 ICK-treated group as compared to the control ICK treated group (p<0.05) at day 83. Although tumor burden (hRLuc activity) was reduced in the CD20-ICK as compared to the control ICK treated group at day 83, no statistical significance was observed. Thus, a trend towards continued T-cell persistence and desired anti-tumor effect in the CD20-ICK treated group was noted.

The above results demonstrate, for the first time, that BLI can be used to connect the persistence of T cells to an anti-tumor effect. These data further reveal that the mice which receive the tumor-specific immunocytokine control their tumor burden to a greater extent than the mice which receive the control immunocytokine (which does not bind the tumor). As a treatment for minimal residual disease in patients undergoing bone marrow transplantation this combination therapy demonstrates the ability to keep the disease relapse in check for almost 3 months in this mouse model.

In aggregate, these data demonstrate that the combination of anti-CD20-IL-2 ICK and CD19R⁺ T cells results in augmented control of tumor growth, as is predicted from the in vivo T-cell persistence data.

It was demonstrated that anti-CD20-IL-2 ICK specifically binds to CD20⁺ tumor, that infusions of the anti-CD204L-2 ICK can augment persistence of adoptively transferred CD19-specific T cells in vivo, and that this leads to improved control of an established CD19⁺CD20⁺ tumor. These observations can be due to the deposition of IL-2 at sites of CD20 binding which provides a positive survival stimulus to infused CD19R⁺IL-2R⁺ effector T cells residing in the tumor microenvironment.

The development of an anti-CD20-IL-2 ICK has implications for future immunotherapy of B-lineage malignancies. While Rituximab® has been extensively used to treat CD20⁺ malignancies (Foran J M, J. Clin. Oncol. (2000) 18:317-24; Maloney D G, et al., Blood 1997; 90:2188-95; Reff M E, et al., Blood (1994) 83:435-45), some patients become unresponsive to this mAb therapy leading to disease progression (McLaughlin P, et al., J. Clin. Oncol. (1998) 16:2825-33). The development of an anti-CD20-IL-2 ICK with its ability to activate immune effector cells, may rescue these patients. Modifications other than the addition of cytokines (Lode H N, Reisfeld R A., Immunol. Res. (2000) 21:279-88; Penichet M L, Morrison S L, J. Immunol. Methods (2001) 248:91-101), such as radionucleotides (Jurcic J G, Scheinberg D A, Curr. Opin. Immunol. ( )1994) 6:715-21), and cytotoxic agents (Kreitman R J, et al., J. Clin. Oncol. (2000) 18:1622-36; Pastan I., Biochim. Biophys. Acta (1997) 1333:1-6), may also improve the therapeutic potential of unconjugated clinical-grade mAbs. Indeed combining mAb-therapy with therapeutic modalities that exhibit non-overlapping toxicity profiles is an attractive strategy to improving the anti-tumor effect without compromising patient safety.

The combination therapy for treating B-lineage tumors described herein combines ICK with T-cell therapy. The two immunotherapies used, anti-CD20-IL-2 ICK and CD19-specific T cells, have the potential to improve the eradication of tumor since (i) the targeting of different cell-surface molecules reduces the possibility emergence of antigen-escape variants, (ii) the mAb conjugated to IL-2 can recruit and activate effector cells (such as CD19-specific T cells) expressing the cytokine receptor in the tumor microenvironment, and (iii) T cells can kill independent of host factors which may limit the effectiveness of mAb-mediated complement dependent cytoxicity (CDC) and antibody dependent cell cytotoxicity (ADCC) (12-15). These immunotherapies will target both malignant and normal B cells. However, as loss of normal B-cell function has not been an impediment to Rituximab® therapy and as clinical conditions associated with hypogammaglobulinemia could be corrected with infusions of exogenous immunoglobulin, a loss of B-cell function may be an acceptable side-effect in patients with advanced B-cell leukemias and lymphomas receiving CD19- and/or CD20-directed therapies.

Another advantage of ICK-therapy is that the loco-regional delivery of T-cell help in the form of IL-2, may avoid the systemic toxicities observed with intravenous infusion of the IL-2 cytokine (43-45) and this may be particularly beneficial in the context of allogeneic hematopoietic stem-cell transplant (HSCT). It has been reported that UCB-derived CD8⁺ T cells can be rendered specific for CD19 to augment the graft-versus-tumor effect after HSCT and since the ICK improves the in vivo immunobiology of UCB-derived CD19-specific T cells, combining the two immunotherapies after UCB transplantation may be beneficial.

Alternative ICK's and T cells with shared specificities for tumor types other than B-lineage malignancies could also be considered for combination immunotherapy. For example, ICK's might be combined with T cells which have been rendered specific by the introduction of chimeric immunoreceptors for breast (46; 47), ovarian (48), colon (49), and brain (50) malignancies. Furthermore, ICK's bearing other cytokines might be infused with T cells to deliver IL-7, IL-15, or IL-21 to further augment T-cell function in the tumor microenvironment.

Currently, the lineage-specific cell-surface molecules CD19 and CD20 present on many B-cell malignancies are targets for both antibody- and cell-based therapies. Coupling these two treatment modalities is predicted to improve the anti-tumor effect, particularly for tumors resistant to single-agent biotherapies. This can be demonstrated using an immunocytokine (ICK), composed of a CD20-specific monoclonal antibody (mAb) fused to biologically-active IL-2, combined with ex vivo-expanded human umbilical cord blood(UCB)-derived CD8⁺ T cells, that have been genetically modified to be CD19-specific, for adoptive transfer after allogeneic hematopoietic stem-cell transplant. It was shown that a benefit of targeted delivery of recombinant IL-2 by the ICK to the CD19⁺CD20⁺ tumor microenvironment is improved in vivo persistence of the CD19-specific T cells and this results in an augmented cell-mediated anti-tumor effect.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed:

1. A device, comprising:
a base unit having a top surface and a bottom surface essentially parallel to and opposite the top surface;
a first reaction tier comprising a plurality of microfluidic chambers impressed into the base unit, each chamber being defined by one or more side walls and a floor and having dimensions that permit the chamber to hold one intact eukaryotic cell; wherein:
each chamber has a port extending from approximately the center of the floor of the chamber to the bottom surface of the base unit, where the port is capable of fluidic connection with an external source; and
each chamber has one or more additional ports extending from the floor of the chamber to the bottom surface of the base unit, where each additional port is individually capable of fluidic connection with an external source;
each chamber has a positive electrode and negative electrode operatively coupled to its wall(s) wherein the electrodes are disposed substantially opposite one another.

2. The device of claim 1, wherein the plurality of microfluidic chambers is divided into arrays of two or more chambers each.

3. The device of claim 1, wherein the center port is operatively coupled to a negative pressure device.

4. The device of claim 1, wherein each port is separated from the chamber by a diffusion barrier.

5. The device of claim 4, wherein the diffusion barrier comprises a mesh having pores about 1 μm in diameter.

6. The device of claim 1, wherein the eukaryotic cell is a primary human T cell.

7. The device of claim 6, wherein each chamber has a volume of about 8000 μm³.

8. The device of claim 2, wherein the arrays of microfluidic chambers are subdivided into two or more subarrays by a wall that surrounds and fluidically separates each subarray from each other subarray thereby forming a second reaction tier.

9. The device of claim 8, wherein the height of the raised walls separating the subarrays is about twice the height of a chamber wall.

10. The device of claim 8, further comprising a second raised wall enclosing all of the subarrays thereby forming a third reaction tier.

11. The device of claim 10, wherein the second raised wall has a wall height of about 2 mm to about 5 mm.

12. The device of claim 2, wherein each array comprises 9 chambers.

13. The device of claim 8, wherein each subarray comprises 9 arrays.

14. The device of claim 13, wherein the total number of chambers is 324.

15. A method of transfecting eukaryotic cells with non-integrating mRNA, comprising:
introducing a plurality of eukaryotic cells into the device of claim 4;
applying a negative pressure through the center port in each chamber;
manipulating the device and cells until one cell enters each chamber and is held there by the applied negative pressure;
removing excess cells;
introducing an electroporation buffer into each chamber;
applying a voltage across the electrodes in each chamber;
introducing an mRNA reagent into each chamber through one of the additional ports in each chamber wherein the mRNA being introduced into each chamber may be the same as or different from the mRNA being introduced into each other chamber;
turning off the voltage across each chamber after a predetermined time;
removing the mRNA reagent from each chamber;
washing the cell in each chamber;
introducing one or more second reagent(s) into each chamber through one or more of the additional ports in each chamber wherein the second reagent(s) being introduced into each chamber may be the same as or different than the second reagent being introduced into each other chamber;
removing the second reagent(s) from each chamber after a second predetermined time;
washing the cells in each chamber;
releasing the negative pressure in those chambers containing similarly treated cells;
optionally applying a positive pressure into each chamber in which the negative pressure has been released through the center port of each chamber;
collecting the released cells; and,
repeating the release of negative pressure and optional application of positive pressure sequentially in chambers holding additional groups of similarly treated cells and collecting the groups of similarly treated cells until all the cells have been collected.

16. The method of claim 15, further comprising:
introducing one or more third reagent(s) into the second reaction tier sub-arrays after removing the second reagent(s) and washing the cells wherein the third reagent(s) introduced into each sub-array may be the same as or different from the third reagent introduced into each other sub-array;
removing the third reagent(s) from the sub-arrays after a third predetermined time;
washing the cells in each chamber;
releasing the negative pressure in those chambers containing cells similarly treated in both the first and second reaction tiers;
optionally applying a positive pressure into each chamber in which the negative pressure has been released through the center port of each chamber;
collecting similarly treated cells; and
repeating the release of negative pressure and optional application of positive pressure sequentially in chambers holding additional groups of similarly treated cells and collecting the groups of similarly treated cells until all the cells have been collected.

17. The method of claim 16, further comprising:
Introducing one or more fourth reagent(s) into the third reaction tier after washing the cells;
removing the fourth reagent(s) from the third reaction tier after a fourth predetermined time;
washing the cells in each chamber;
releasing the negative pressure in those chambers containing cells similarly treated in the first, second and third reaction tiers;
optionally applying a positive pressure into each chamber in which the negative pressure has been released through the center port of each chamber;
collecting similarly treated cells; and
repeating the release of negative pressure and optional application of positive pressure sequentially in chambers holding additional groups of similarly treated cells and collecting the groups of similarly treated cells until all the cells have been collected.

18. A device comprising:
an orifice plate having an inlet surface, an outlet surface and an outer edge having a thickness;
one or more through-holes extending through the orifice plate from the inlet surface to the outlet surface, the surface between the inlet and outlet surfaces comprising a wall surface; wherein
each through-hole is sized to permit a single eukaryotic cell at a time pass through;
each through-hole has a positive electrode operatively coupled to its wall surface substantially opposite a negative electrode likewise operatively coupled to its wall surface;
a positive electrode connection and an negative electrode connection operatively coupled to the outer edge of the orifice plate, the positive electrode connection being operatively coupled to each positive electrode in each through-hole and the negative electrode connection being operatively coupled to each negative electrode in each through-hole;
an inlet exterior source connector operatively coupled to the inlet surface of the orifice plate; and
an outlet connector operatively coupled to the outlet surface of the orifice plate.

19. The device of claim 18, further comprising two or more external sources operatively coupled to the inlet exterior course connector.

20. The device of claim 19, where one external source is a source of eukaryotic cells and another external source is a source of a non-integrating nucleic acid.

21. The device of claim 20, wherein the eukaryotic cells are primary human T-cells.

22. The device of claim 20, wherein the non-integrating nucleic acid is non-integrating mRNA.

23. The device of claim 18, wherein the outlet connector is operatively coupled to a collection device.

24. The device of claim 18, further comprising a u-shaped construct having a base and two side parallel side walls, one side wall having a positive pole electrical contact operatively coupled to a positive pole of an external voltage source and the other side wall having a negative pole electrical contact operatively coupled to a negative pole of the external voltage source, wherein
the side walls are spaced apart such that when the orifice plate is placed between them the positive electrode connection makes electrical contact with the positive pole electrical contact on one wall of the U-shaped construct and the negative electrode connection makes electrical contact with the negative pole electrical contact on the opposite wall of the U-shaped construct.

25. A method of treating a disease, comprising:
identifying a subject afflicted with a disease that is known to be, becomes known to be or is suspected of being responsive to treatment using transfected cells;
inserting a sterile needle that is operatively coupled to a cell separator that in turn is operatively coupled to the inlet exterior source connector of the device of claim 17 into a blood vessel of a subject;
withdrawing blood from the subject and transporting it through sterile tubing to the cell separator wherein cells of a type that is to be electro-transfected are selected and separated from other cell types in the blood;
introducing the selected cells along with a non-integrating nucleic acid to the input surface side of the orifice plate and then passing the mixture through the through-holes in the orifice plate in which through-holes a voltage has been created using the external voltage source such that the cells are electroporated and transfected as they pass through;
transporting the transfected cells through the outlet connector, which has been operatively connected to a sterile syringe needle that has been inserted into a blood vessel of the subject, back into the subject.

26. The method of claim 25, wherein the subject is a mammal.

27. The method of claim 26, wherein the mammal is a human being.

28. The method of claim 27, wherein the human being is a pediatric patient.

29. The method of claim 25, wherein the selected cell type is selected from the group consisting of T cells, NK cells, B cells, dendritic (antigen presenting) cells, monocytes, reticulocytes, stem cells, tumor cells, umbilical cord blood-derived cells, peripheral-blood derived cells and combinations thereof.

30. The method of claim 29, wherein the stems cells are selected from the group consisting of hematopoitic stem cells and mesenchymal stem cells.

31. The method of claim 29, wherein the selected cell type is selected from the group consisting of T cells, NK cells or a combination thereof.

32. The method of claim 25, wherein the selected cell type is primary human T-cells.

33. The method of claim 25, wherein the non-integrating nucleic acid is a non-integrating RNA.

34. The method of claim 33, wherein the non-integrating RNA is selected from the group consisting of mRNA, microRNA and siRNA.

35. The method of claim 34, wherein the non-integrating RNA codes for a biotherapeutic agent.

36. The method of claim 35, wherein the biotherapeutic agent is selected from the group consisting of a chimeric antigen receptor, an enzyme, a hormone, an antibody, a clotting factor, a Notch ligand, a recombinant antigen for vaccine, a cytokine, a cytokine receptor, a chemokine, a chemokine receptor, an imaging transgene, a co-stimulatory molecule, a T-cell receptor, FoxP3, a luminescent probe, a fluorescent probe, a reporter probe for positron emission tomography, a sodium iodine symporter, a KIR deactivator, hemoglobin, an Fc receptor, CD24, BTLA, a transposase, a transposon, a transposon from Sleeping Beauty or piggyback and combinations thereof.

37. The method of claim 25, wherein the disease is selected from the group consisting of a pathogenic disorder, cancer, enzyme deficiency, in-born error of metabolism, infection, auto-immune disease, obesity, cardiovascular disease, neurological disease, neuromuscular disease, blood disorder, clotting disorder and a cosmetic defect.
